(19) United States
(12) Patent Application Publication
(10) Pub. No.: US 2010/0292184 A1
(43) Pub. Date: Nov. 18, 2010

(54) 17BETA-CYANO-19-ANDROST-4-ENE DERIVATIVE, ITS USE AND MEDICAMENTS COMPRISING THE DERIVATIVE

(75) Inventors: Joachim Kuhnke, Potsdam (DE); Jan Huebner, Berlin (DE); Rolf Bohlmann, Berlin (DE); Thomas Frenzel, Hofheim (Taunus) (DE); Ulrich Klar, Berlin (DE); Frederik Menges, Schriesheim (DE); Sven Ring, Jena (DE); Steffen Borden, Berlin (DE); Hans Peter Muhn, Berlin (DE); Katja Prelle, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: BAYER SCHERING PHARMA AG, Berlin (DE)

(21) Appl. No.: 12/664,099

(22) PCT Filed: Jun. 12, 2008
(86) PCT No.: PCT/EP2008/057427

§ 371 Date: Apr. 20, 2010

Related U.S. Application Data
(60) Provisional application No. 60/943,651, filed on Jun. 13, 2007.

(30) Foreign Application Priority Data
Jun. 12, 2007 (DE) 10 2007 027 635.6

(51) Int. Cl. A61K 31/56; C07J 53/00; C07J 41/00; A61K 31/695; A61K 31/565; A61P 15/18; A61P 5/24; A61P 5/34; A61P 5/42

(52) U.S. Cl. 514/63; 552/510; 552/513; 552/611; 552/508; 552/505; 514/177; 514/170

(57) ABSTRACT

The 17β-cyano-19-androst-4-ene derivatives of the present invention possess gestagenic activity. They have the general chemical formula 1, in which Z is selected from the group comprising O, two hydrogen atoms, NOR and NNHSO₂R, in which R is hydrogen or C₁-C₄-alkyl, R¹, R² are each independently hydrogen or methyl, or R¹ and R² together form methylene or are omitted with formation of a double bond between C¹ and C², R⁴ is hydrogen or halogen, furthermore either: R^{6a}, R^{6b} together form methylene or 1,2-ethanediyl or R^{6a} is hydrogen and R^{6b} is selected from the group comprising hydrogen, methyl and hydroxymethylene, and R⁷ is selected from the group comprising hydrogen, C₁-C₄-alkyl, C₂-C₃-alkenyl and cyclopropyl, or: R^{6a} hydrogen and R^{6b} and R⁷ together form methylene or are omitted with formation of a double bond between C⁶ and C⁷ or: R^{6a} is methyl and R^{6b} and R⁷ are omitted with formation of a double bond between C⁶ and C⁷, R¹⁵, R¹⁶ are hydrogen or together form methylene, R¹⁷ is selected from the group comprising hydrogen, C₁-C₄-alkyl and allyl, and moreover comprise their solvates, hydrates, stereoisomers, diastereomers, enantiomers and salts, with the proviso that particular compounds are excluded.

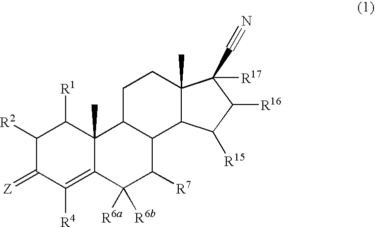

The invention relates to certain 17β-cyano-19-androst-4-ene derivatives, their use and to medicaments comprising the derivatives and having gestagenic action, for example for the treatment of pre-, perk and postmenopausal symptoms and of premenstrual symptoms.

From the literature, compounds having gestagenic, antimineralcorticoid, antiandrogenic or antioestrogenic action based on a steroid structure are known, which are derived, for example, from 19-androst-4-en-3-one or a derivative thereof (the numbering of the steroid structure can be taken, for example, from Fresenius/Görlitzer 3rd ed. 1991 “Organisch-chemische Nomenklatur” [Organic chemical nomenclature] pp. 60 ff.).

Thus, WO 2006072467 A1 describes the compound 6β,7β-15β,16β-dimethylene-3-oxo-17-pregn-4-ene-21,17β-carbolactone (drospirenone) having gestagenic action, which has been used, for example, in an oral contraceptive and a preparation for the treatment of postmenopausal symptoms. On account of its comparatively low affinity for the gestagen receptor and its comparatively high ovulation-inhibiting dose, drospirenone is contained in the contraceptive, however, in the relatively high daily dose of 3 mg. Drospirenone is moreover distinguished in that, in addition to the gestagenic action, it has aldosterone-antagonistic (antimineralcorticoid) and antiandrogenic action. These two properties make drospirenone very similar in its pharmacological profile to the natural gestagen progesterone which, however, unlike drospirenone is not adequately bioavailable orally. In order to lower the dose to be administered, in WO 2006072467 A1 an 18-methyl-19-nor-17-pregn-4-ene-21,17-carbolactone and pharmaceutical preparations comprising this are further proposed which have a higher gestagenic potency than drospirenone.

In addition, for example, U.S. Pat. No. 3,705,179 discloses steroids which have antiandrogenic activity and are suitable for the treatment of illnesses which are connected with androgens. Among other compounds, 17β-cyano-17α-methylandrost-4-en-3-one derivatives are disclosed.

In DE 22 26 552 B2, further 17-cyano-19-nor-androst-4-en-3-one compounds are described which show progestomimetic, antiandrogenic and antioestrogenic actions having exogenous character.

The object of the present invention is to make available compounds which have strong binding to the gestagen receptor. Moreover, the compounds should preferably also have an antimineralcorticoid action.

This object was achieved by the novel 17β-cyano-19-androst-4-ene derivatives according to Claim 1, the use of the novel derivatives according to Claim 15, and a medicament comprising at least one novel derivative according to Claim 17. Advantageous embodiments of the invention are indicated in the subclaims.

The present invention accordingly relates to a 17β-cyano-19-androst-4-ene derivative having the general chemical formula 1

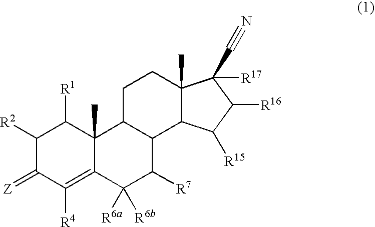

where
Z is selected from the group comprising O, two hydrogen atoms, NOR and NNHSO₂R, in which R is hydrogen or C₁-C₄-alkyl, R¹, R² are each independently hydrogen or methyl, or R¹ and R² together form methylene or are omitted with formation of a double bond between C¹ and C²,
R⁴ is hydrogen or halogen,
furthermore either:
R^{6a}, R^{6b} together form methylene or 1,2-ethanediyl or R^{6a} is hydrogen and R^{6b} is selected from the group comprising hydrogen, methyl and hydroxymethylene, and
R⁷ is selected from the group comprising hydrogen, C₁-C₄-alkyl, C₂-C₃-alkenyl and cyclopropyl,
or:
   R^{6a} is hydrogen and R^{6b} and R⁷ together form methylene or are omitted with formation of a double bond between C⁶ and C⁷
or:
   R^{6a} is methyl and R^{6b} and R⁷ are omitted with formation of a double bond between C⁶ and C⁷,
R¹⁵, R¹⁶ are hydrogen or together form methylene,
R¹⁷ is selected from the group comprising hydrogen, C₁-C₄-alkyl and allyl,
and its solvates, hydrates, stereoisomers, diastereomers, enantiomers and salts,
with the proviso that compounds with the following general chemical formula A are excluded:

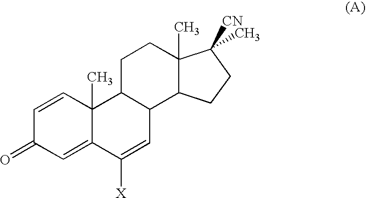

in which X is hydrogen or methyl and the double bonds between C¹ and C² and between C⁶ and C⁷ are optional double bonds and
with the further proviso that 17β-cyanoandrost-4-en-3-one is also excluded.

The compounds according to the general chemical formula A excluded from the present invention are the following compounds:

| Compounds excluded | X | C¹═C² | C⁶═C⁷ |
|---|---|---|---|
| 17β-Cyano-17α-methylandrost-4-en-3-one | ./. | ./. | ./. |
| 17β-Cyano-6α,17α-dimethylandrost-4-en-3- | + | ./. | ./. |
| one | | | |
| 17β-Cyano-17α-methylandrosta-1,4-dien-3-one | ./. | + | ./. |
| 17β-Cyano-6α,17α-dimethylandrosta-1,4-dien- | + | + | ./. |
| 3-one | | | |
| 17β-Cyano-17α-methylandrosta-4,6-dien-3-one | ./. | ./. | + |
| 17β-Cyano-6α,17α-dimethylandrosta-4,6-dien- | + | ./. | + |
| 3-one | | | |
| 17β-Cyano-17α-methylandrosta-1,4,6-trien-3- | ./. | + | + |
| one | | | |
| 17β-Cyano-6α,17α-dimethyl-1,4,6-trien-3-one | + | + | + |

“./.” not present;
“+”: present

The numbering of the C ring system of the novel derivative of the general chemical formula 1 customarily follows the numbering of a steroid ring system, described, for example, in Fresenius, loc. cit. The numbering of the radicals indicated in the claims analogously corresponds to their bonding position to the C ring system of the derivative. For instance, the radical R⁴ bonds to the C⁴-position of the novel derivative.

With respect to the groups defined for Z, the groups NOR and NNHSO₂R in each case bond using a double bond via N to the C skeleton of the derivative as in ═NOR and ═N—NH—SO₂R. OR in NOR and NHSO₂R in NNHSO₂R can be in the syn or anti position.

C₁-C₄-Alkyl is in each case understood as meaning a straight-chain or branched alkyl radical, for example methyl, ethyl, n-propyl, isopropyl, n-butyl, isobutyl or tent-butyl, especially the unbranched radicals. Methyl, ethyl and n-propyl are particularly preferred. Alkyl radicals bonded in the 17α position can moreover be perfluorinated, such that R¹⁷ in this case can moreover be trifluoromethyl, pentafluoroethyl, n-heptafluoropropyl, isoheptafluoropropyl, n-nonafluorobutyl, isononafluorobutyl and tert-nonafluorobutyl.

C₂-C₃-Alkenyl is preferably to be understood as meaning vinyl or allyl.

Halogen is in each case to be understood as meaning fluorine, chlorine, bromine or iodine.

Isomers are chemical compounds having the same empirical formula, but different chemical structure. Expressly, all possible isomers and isomer mixtures (racemates) are additionally included, the 17β-cyano position being specified in the novel derivative.

In general, constitutional isomers and stereoisomers are differentiated. Constitutional isomers have the same empirical formula, but differ in the manner of linkage of their atoms or atomic groups. These include functional isomers, positional isomers, tautomers or valence isomers. In principle, stereoisomers have the same structure (constitution) and thus also the same empirical formula, but differ in the spatial arrangement of the atoms. In general, configurational isomers and conformational isomers are differentiated. Configurational isomers are stereoisomers which can only be converted into one another by bond breakage. These include enantiomers, diastereomers and E/Z (cis/trans) isomers. Enantiomers are stereoisomers which behave as image and mirror image to one another and have no plane of symmetry. All stereoisomers which are not enantiomers are designated as diastereomers. E/Z (cis/trans) isomers on double bonds are a special case. Conformational isomers are stereoisomers which can be converted into one another by the rotation of single bonds. For the delineation of the types of isomerism from one another see also the IUPAC rules, section E (Pure Appl. Chem. 45, 11-30 (1976)).

The novel derivatives having the general chemical formula 1 also comprise the possible tautomeric forms and include the E or Z isomers or, if a chiral centre is present, also the racemates and enantiomers. Double bond isomers are also to be understood among these.

The novel derivatives can also be present in the form of solvates, in particular of hydrates, the novel compounds accordingly containing polar solvents, in particular water, as a structural element of the crystal lattice of the novel compounds. The polar solvent, in particular water, can be present in a stoichiometric or alternatively unstoichiometric ratio. In the case of stoichiometric solvates, hydrates, hemi-, (semi-), mono-, sesqui-, di-, tri-, tetra-, penta-, etc. solvates or hydrates are also spoken of.

It has been found that the novel compounds or derivatives have a good gestagenic action in vivo. Moreover, some interesting novel compounds act as antagonists for the mineralcorticoid receptor.

Novel derivatives having the aforementioned general chemical formula 1 are preferred in which Z is selected from the group comprising O, NOH and NNHSO₂H. Z is particularly preferably O.

Independently of the selection of Z, novel derivatives having the aforementioned general chemical formula 1 are furthermore preferred in which the following variants occur alternatively or else at least in some cases together and are selected independently of one another:

R¹⁵ and R¹⁶ especially preferably together form methylene, where both an α- and a β-methylene group can be bonded in these positions.

R¹ and R² are furthermore preferably each hydrogen or together form methylene, particularly preferably α-methylene. R¹ is more preferably α-methyl.

R⁴ is furthermore preferably hydrogen or chlorine.

R^{6a} and R^{6b} furthermore preferably together form 1,2-ethanediyl or are in each case hydrogen.

R⁷ is furthermore preferably selected from the group comprising hydrogen and methyl, where the methyl group can be both α- and β-.

R^{6b} and R⁷ furthermore preferably together form methylene, where the methylene group can be both α- and β-.

R¹⁷ is furthermore preferably selected from the group comprising hydrogen and methyl.

The radicals R^{6a}, R^{6b}, R⁷, R¹⁵ and R¹⁶ can furthermore be both α- and β-.

The novel 17β-cyano-19-nor-androst-4-ene derivatives are particularly preferably selected from the group comprising:

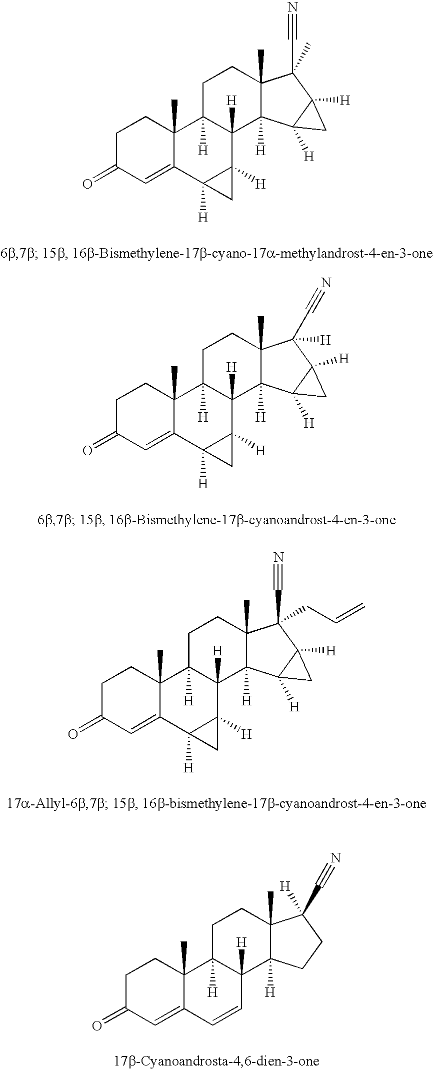

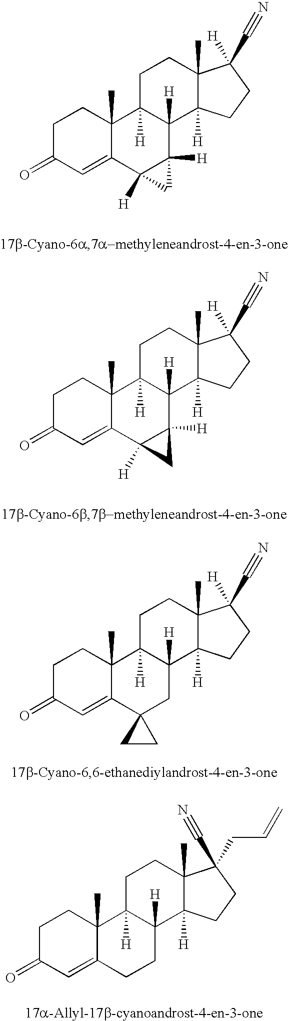

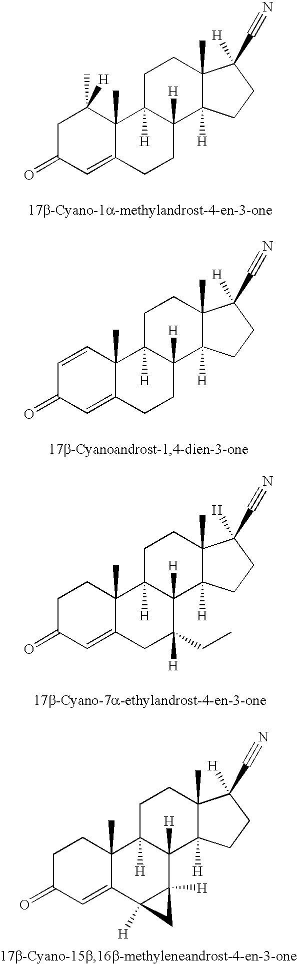

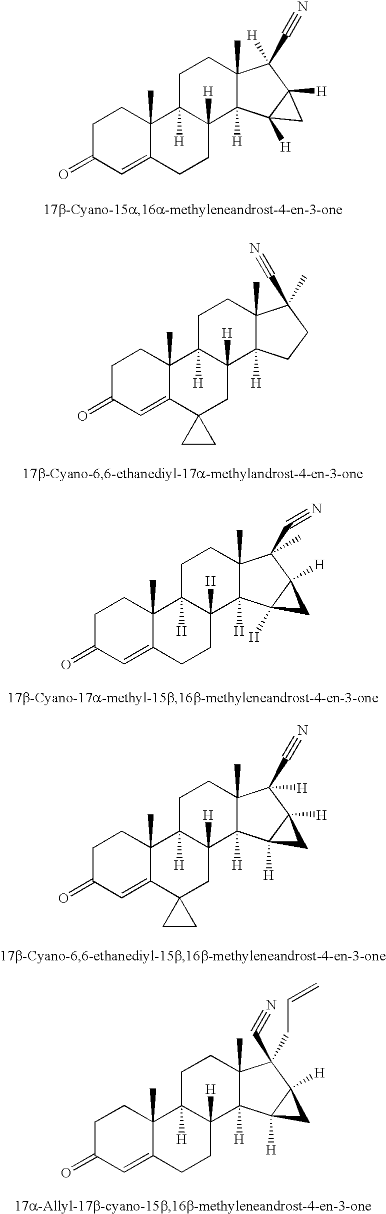

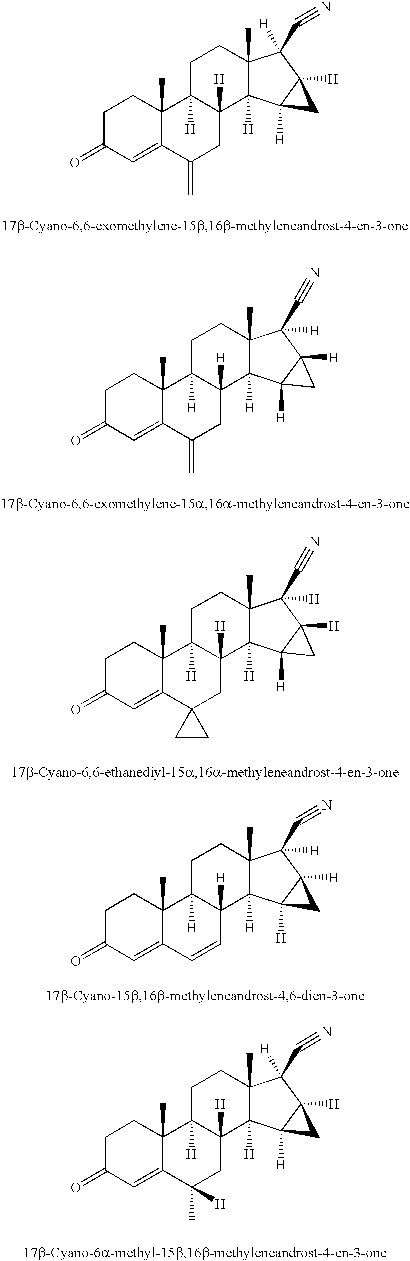

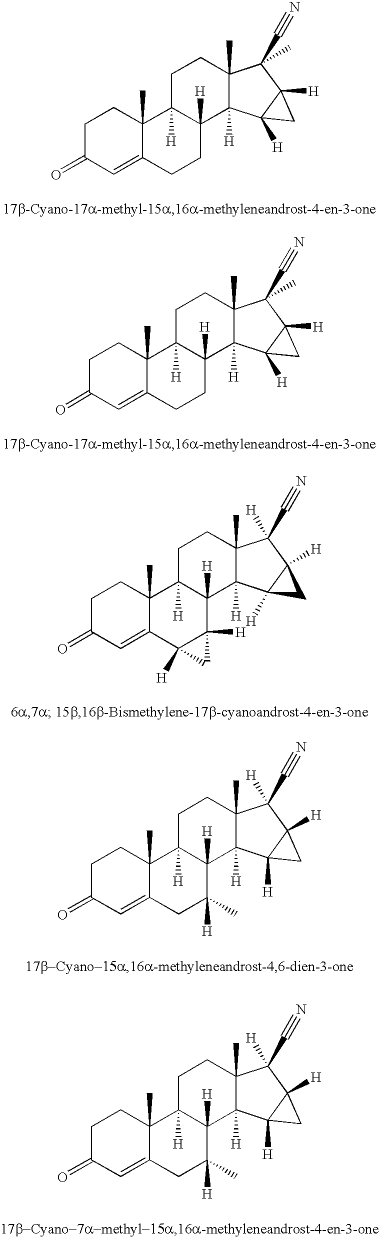

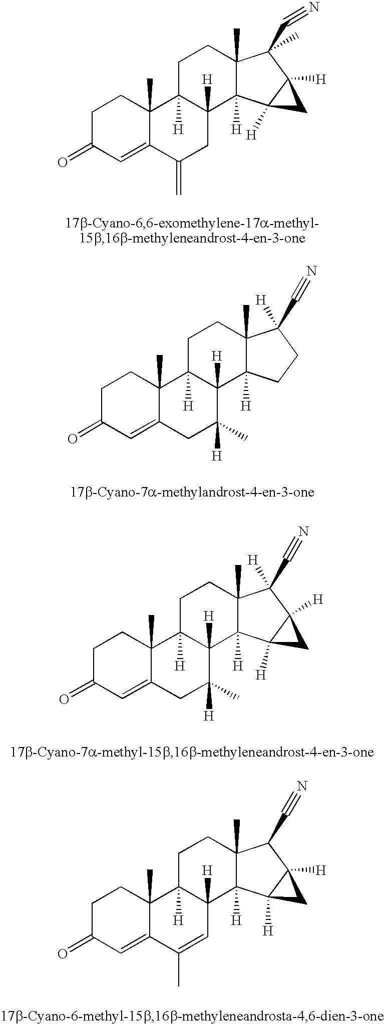

The 15α,16α- and the 15β,16β-methylene derivatives in the above list are very particularly preferred.

On account of their gestagenic activity, the novel compounds having the general chemical formula 1 can be used alone or in combination with oestrogens in medicaments for contraception.

The derivatives according to the invention are therefore suitable in particular for the production of a medicament for oral contraception and for the treatment of pre-, peri- and postmenopausal symptoms, including use in preparations for hormone replacement therapy (HRT).

Because of their favourable profile of action, the derivatives according to the invention are particularly highly suitable for the treatment of premenstrual symptoms, such as headaches, depressive moods, water retention and mastodynia.

The use of the derivatives according to the invention for the production of a medicament having gestagenic and antimineralcorticoid action is particularly preferred.

Treatment with the derivatives according to the invention preferably takes place in humans, but can also be carried out on related mammalian species, such as, for example, on dog and cats.

For the use of the derivatives according to the invention as medicaments, these are combined with at least one suitable pharmaceutically harmless additive, for example vehicle. The additive is suitable, for example, for parenteral, preferably oral, administration. It is a matter here of pharmaceutically suitable organic or inorganic inert additive materials, such as, for example, water, gelatine, gum arabicum, lactose, starch, magnesium stearate, talc, vegetable oils, polyalkylene glycols etc. The medicaments can be present in solid form, for example as tablets, coated tablets, suppositories, capsules, or in liquid form, for example as solutions, suspensions or emulsions. Optionally, they moreover contain excipients, such as preservatives, stabilizers, wetting agents or emulsifiers, salts for changing the osmotic pressure or buffers. For parenteral administration, oily solutions, such as, for example, solutions in sesame oil, castor oil and cottonseed oil, are in particular suitable. To increase the solubility, solubilizers, such as, for example, benzyl benzoate or benzyl alcohol, can be added. It is also possible to incorporate the derivatives according to the invention into a transdermal system and thus to administer it transdermally. For oral administration, tablets, coated tablets, capsules, pills, suspensions or solutions are in particular suitable.

The dose of the derivatives according to the invention in contraception preparations should be 0.01 to 10 mg per day. The daily dose in the case of the treatment of premenstrual symptoms is approximately 0.1 to 20 mg. The gestagenic derivatives according to the invention are preferably administered orally in contraception preparations and in the medicaments for the treatment of premenstrual symptoms. The daily dose was preferably administered as a single dose.

The gestagenic and oestrogenic active substance components are preferably administered together orally in contraception preparations. The daily dose was preferably administered as a single dose.

Possible oestrogens are synthetic oestrogens, preferably ethinylestradiol, but also mestranol.

The oestrogen was administered in a daily amount which corresponds to that of 0.01 to 0.04 mg of ethynylestradiol.

Oestrogens, of course, are primarily used as oestrogens in the medicaments for the treatment of pre-, peri- and postmenopausal symptoms and for hormone replacement therapy, especially oestradiol or its esters, for example oestradiol valerate, or alternatively conjugated oestrogens (CEEs=Conjugated Equine Estrogens).

If the preparation of the starting compounds is not described here, these are known to the person skilled in the art or can be prepared analogously to known compounds or processes described here. The isomer mixtures can be separated into the enantiomers, E/Z isomers or epimers by customary methods, such as, for example, crystallization, chromatography or salt formation.

The derivatives according to the invention having the general chemical formula 1 are prepared as described below.

Suitable starting materials for the 17β-cyanoandrost-4-en-3-one derivatives described here are various steroidal starting materials, such as, for example, androst-4-ene-3,17-dione (see, for example, J. Am. Chem. Soc. 87, 3727 (1965)), or the partially reduced analogues, such as testosterone or else prasterone.

Suitable starting materials which bear a 15α,16α- or else 15β,16β-methylene group are likewise known from the literature (e.g. 15α,16α-methyleneandrost-5-en-17-on-3β-ol; see Chem. Bar. 106, 888 (1973); the corresponding Δ4-3,17-dione; see DE-A 21 09 555 (1972). The 15β,16β-methyleneandrost-4-ene-3,17-dione is described in Izv. Nauk SSSR Ser. Khim. 8, 1893 (1985) and in Chem. Ber. 107, 128-134 (1974); the corresponding Δ5-3-alcohol in Angew. Chem. 94 (9), 718 (1982). It is obvious to the person skilled in the art that in the descriptions of the synthetic transformations it was always provided for other functional groups optionally present on the steroid ring system to be protected in suitable form.

The introduction of a nitrile into position 17 (C¹⁷) of the steroid ring system can be carried out in a variety of ways. Both single-stage processes and multistage variants are possible here. Methods are preferred here which finally mean the replacement of an oxygen function by cyanide. Many possible process variants are described in Science of Synthesis Houben-Weyl Methods of Molecular Transformations Category 3 Volume 19 pp. 197-213 (2004 Georg Thieme Verlag Stuttgart, New York) and in Houben-Weyl Methadon der organischen Chemie [Houben-Weyl Methods of organic chemistry] Volume E5 Part 2 pp. 1318-1527 (1985 Georg Thieme Verlag Stuttgart, New York).

A single-stage process which suggests itself is, for example, the direct reductive replacement of a carbonyl oxygen atom by a cyano group. For this, a 17-ketosteroid was reacted with tosylmethyl isocyanide in suitable solvents, such as, for example, dimethoxyethane, dimethyl sulphoxide, ethers, alcohols or alternatively their mixtures, using suitable bases, such as, for example, alkali metal alkoxides, alkali metal hydrides, potassium hexamethyldisilazide, or alternatively alkali metal amides, such as, for example, lithium diisopropylamide, in a temperature range from 0° C. to 100° C. 17-Epimer mixtures which may be formed can be separated by chromatography, fractional crystallization or using a combination of these methods.

The SN₂-type replacement of a suitable leaving group in position 17, such as, for example, of a halide (preferably iodine or bromine), or alternatively of a sulphonic acid ester of a 17-alcohol, by cyanide is also possible. Cyanide sources used are preferably inorganic cyanides, such as lithium cyanide, sodium cyanide and potassium cyanide.

The following may be mentioned as examples of multistage variants of nitrile introduction: a 17-ketone was converted by means of a Wittig olefination to the corresponding 17-exomethylene compound, which after hydroboration and oxidation to the aldehyde can be reacted to give the corresponding 17-carbaldehyde oxime. Dehydration of the oxime then leads to the 17-nitrile.

The introduction of the nitrile can be carried out both at the beginning of a synthesis sequence and also at any desired later point in time, provided that further functional groups which may be present are protected in a suitable manner.

The 17-cyano compounds can be optionally alkylated, which leads to stereochemically homogeneous 17β-cyano-17α-substituted derivatives. For this, the 17-cyanosteroid was deprotonated in a suitable solvent, such as, for example, ethers, for example tetrahydrofuran. Various bases can be used here, for example an alkali metal amide, such as lithium diisopropylamide. After addition of an alkylating agent, such as, for example, of an alkyl or alkenyl halide, and work-up, the 17β-cyano-17α-substituted derivatives are then obtained.

By way of example, the further synthetic procedure may be illustrated with the aid of the following synthesis scheme, the compound 2 (Bull. Soc. Chim. Fr. 1835 (1976); U.S. Pat. No. 3,705,179 (1971)) already described being mentioned as a starting material:

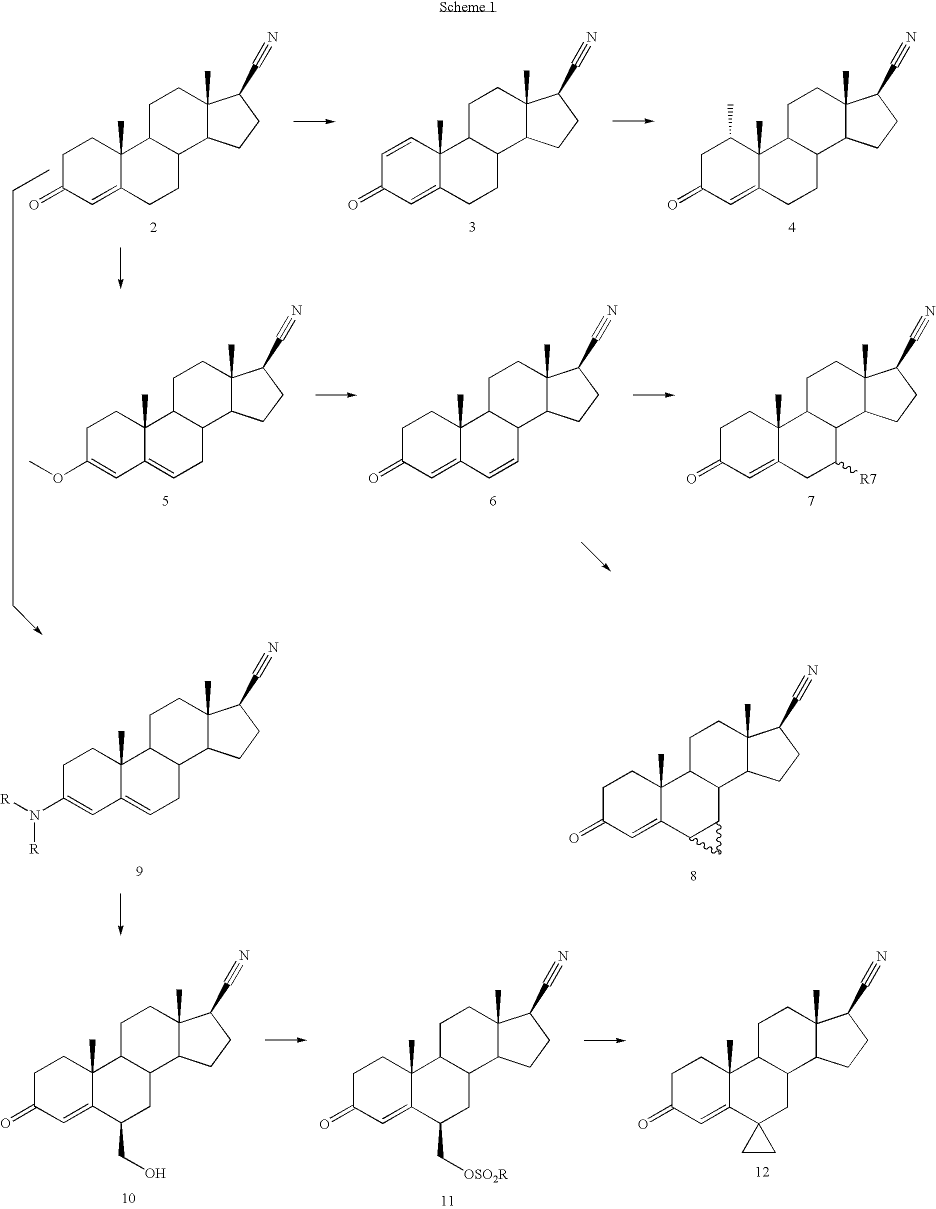

The introduction of a 1,2-double bond into the compound 2 then leads to 3. Possible dehydrating agents here, among others, are selenium dioxide (J. Org. Chem. 21, 239 (1956)) or else 2,3-dichloro-5,6-dicyanobenzoquinone (Steroids 35 (5), 481 (1980)). A 1,4-addition to the 1-methyl derivative 4 can be performed, for example, with trimethylaluminium with addition of trimethylsilyl chloride and copper bromide in suitable solvents (Angewandte 105 (9), 1429 (1993)).

The introduction of a 6,7-double bond is carried out by means of bromination of the 3,5-dienol ether 5 and subsequent elimination of hydrogen bromide (see, for example, J. Fried, J. A. Edwards, Organic Reactions in Steroid Chemistry, von Nostrand Reinhold Company 1972, pp. 265-374).

The introduction of a substituent R⁴ can be achieved, for example, starting from a compound of the formula 2, by epoxidation of the 4,5-double bond with hydrogen peroxide under alkaline conditions and reaction of the resulting epoxides in a suitable solvent with acids having the general chemical formula H—R⁴, where R⁴ can be a halogen atom or a pseudohalogen, or by reacting with catalytic amounts of mineral acid and optionally reacting the 4-bromo compounds obtained having the general chemical formula 1 (where R⁴=bromine) with methyl 2,2-difluoro-2-(fluorosulphonyl)-acetate in dimethylformamide in the presence of copper(I) iodide.

The dienol ether bromination of compound 5 can be carried out, for example, analogously to the procedure of Steroids 1, 233 (1963). The elimination of hydrogen bromide is possible by heating the 6-bromo compound with basic reagents, such as, for example, LiBr or Li₂CO₃, in aprotic solvents, such as dimethylformamide, at temperatures from 50° C. to 120° C. or else by heating the 6-bromo compounds in a solvent, such as collidine or lutidine, to give compound 6.

Compound 7 is converted by methenylation of the 6,7-double bond according to known processes, for example using dimethylsulphoxonium methylide (see, for example, DE-A 11 83 500, DE-A 29 22 500, EP-A 0 019 690, U.S. Pat. No. 4,291,029; J. Am. Chem. Soc. 84, 867 (1962)) to a compound 8, a mixture of the α- and β-isomers being obtained, which can be separated into the individual isomers, for example, by chromatography.

Compounds of the type 7 can be obtained as described in the examples or analogously to these procedures using reagents analogous to those described there.

The synthesis of the spirocyclic compound 12 starts from 2, which was first converted to a 3-amino-3,5-diene derivative 9. By reaction with formalin in alcoholic solution, the 6-hydroxymethylene derivative 10 was obtained. After conversion of the hydroxyl group to a leaving group, such as, for example, a mesylate, tosylate (compound 11) or alternatively benzoate, compound 13 can be prepared by reaction with trimethylsulphoxonium iodide using bases, such as, for example, alkali metal hydroxides or alkali metal alkoxides, in suitable solvents, such as, for example, dimethyl sulphoxide.

For the introduction of a 6-methylene group, compound 10 can be dehydrated using, for example, hydrochloric acid in dioxane/water. 6-Methylene can also be produced from 11 (see DE-A 34 02 3291, EP-A 0 150 157, U.S. Pat. No. 4,584,288; J. Med. Chem. 34, 2464 (1991)).

A further possibility for the preparation of 6-methylene compounds consists in the direct reaction of the 4(5) unsaturated 3-ketones, such as compound 2, with acetals of formaldehyde in the presence of sodium acetate using, for example, phosphorus oxychloride or phosphorus pentachloride in suitable solvents, such as chloroform (see, for example, K. Annen, H. Hofineister, H. Laurent and R. Wiechert, Synthesis 34 (1982)).

The 6-methylene compounds can be used for the preparation of compounds having the general formula 1, in which R^{6a} is equal to methyl and R^{6b} and R⁷ are omitted with formation of a double bond between C⁶ and C⁷.

For this, for example, a process described in Tetrahedron 21, 1619 (1965) can be used, in which an isomerization of the double bond is achieved by warming the 6-methylene compounds in ethanol with 5% palladium-carbon catalyst, which was pretreated either with hydrogen or by warming with a small amount of cyclohexene. The isomerization can also be carried out using a catalyst which was not pretreated, if a small amount of cyclohexene was added to the reaction mixture. The occurrence of small amounts of hydrogenated products can be prevented by addition of an excess of sodium acetate.

The 6-methyl-4,6-dien-3-one derivatives, however, can also be prepared directly (see K. Annen, H. Hofineister, H. Laurent and R. Wiechert, Lieb. Ann. 712 (1983)).

Compounds in which R^{6b} is an α-methyl function can be prepared from the 6-methylene compounds by hydrogenation under suitable conditions. The best results (selective hydrogenation of the exomethylene function) are achieved by transfer hydrogenation (J. Chem. Soc. 3578 (1954)). If the 6-methylene derivatives are heated in a suitable solvent, such as, for example, ethanol, in the presence of a hydride donor, such as, for example, cyclohexene, 6α-methyl derivatives are obtained in very good yields. Small amounts of 6β-methyl compound can be isomerized by acid (Tetrahedron 1619 (1965)).

The selective preparation of 6β-methyl compounds is also possible. For this, the 4-en-3-ones, such as, for example, compound 2, are reacted, for example, with ethylene glycol or trimethyl orthoformate in dichloromethane in the presence of catalytic amounts of an acid, e.g. p-toluenesulphonic acid, to give the corresponding 3-ketals. During this ketalization, the double bond in position 5 (C⁵) isomerizes. A selective epoxidation of this 5-double bond is possible, for example, by use of organic peracids, e.g. of m-chloroperbenzoic acid, in suitable solvents, such as dichloromethane. Alternatively to this, the epoxidation can also be carried out using hydrogen peroxide in the presence of, for example, hexachloroacetone or 3-nitrotrifluoroacetophenone. The 5,6α-epoxides can then be opened axially using appropriate alkylmagnesium halides or alkyllithium compounds. 5α-Hydroxy-6β-alkyl compounds are thus obtained. The cleavage of the 3-keto protective group can be carried out with obtainment of the 5α-hydroxyl function by treating under mild acidic conditions (acetic acid or 4 N hydrochloric acid at 0° C.). Basic elimination of the 5α-hydroxyl function using, for example, diluted aqueous sodium hydroxide solution affords the 3-keto-4-ene compounds having a β-6-alkyl group. Alternatively to this, ketal cleavage under more drastic conditions (aqueous hydrochloric acid or another strong acid) affords the corresponding 6α-alkyl compounds.

The compounds having the general chemical formula 1 obtained, in which Z is an oxygen atom, can be converted to their corresponding oximes (general chemical formula 1 with Z denoting NOH, where the hydroxyl group can be syn- or anti-) by reaction with hydroxylamine hydrochloride in the presence of a tertiary amine at temperatures between −20 and +40° C. Suitable tertiary bases are, for example, trimethylamine, triethylamine, pyridine, N,N-dimethylaminopyridine, 1,5-diazabicyclo[4.3.0]non-5-ene (DBN) and 1,5-diazabicyclo[5.4.0]undec-5-ene (DBU), pyridine being preferred. This applies analogously as is described in WO-A 98/24801 for the preparation of corresponding 3-oxyimino derivatives of drospirenone.

The removal of the 3-oxo group for the preparation of a final product having the general chemical formula 1 with Z denoting two hydrogen atoms can be carried out, for example, by reductive cleavage of a thioketal of the 3-keto compound according to the procedure indicated in DE-A 28 05 490.

The following examples serve for the more detailed illustration of the invention:

The compounds according to the invention are surprisingly distinguished by strong gestagenic activity and are strongly active in the maintenance of pregnancy test on the rat after subcutaneous administration.

Carrying out the maintenance of pregnancy test on the rat:

In pregnant rats, the removal of the corpora lutea or oophorectomy induces an abortion. By means of the exogenous administration of progestins (gestagens) in combination with a suitable dose of an oestrogen, the maintenance of pregnancy is possible. The maintenance of pregnancy test on ovarectomized rats serves for the determination of the peripheral gestagenic activity of a compound.

Rats were paired overnight during proestrus. Pairing was checked on the morning of the following day by the appraisal of a vaginal smear. The presence of the sperm was evaluated here as day 1 of a commencing pregnancy. On day 8 of the pregnancy, the animals were ovarectomized under ether anaesthesia. The treatment with test compound and exogenous oestrogen (oestrone, 5 μg/kg/day) was carried out subcutaneously once daily from day 8 to day 15 or day 21 of the pregnancy. The first administration on day 8 was carried out two hours before oophorectomy. Intact control animals were given exclusively vehicle.

### Evaluation:

At the end of the experiment (day 15 or day 21), the animals were sacrificed under a CO₂ atmosphere, and live foetuses (foetuses having a beating heart) and implantation sites (early resorptions and dead foetuses including autolysis and atrophic placentas) were counted in both uterine horns. On day 22, it was moreover possible to examine foetuses for malformations. In uteri without foetuses or implantation sites, the number of nidation sites was determined by staining with 10% strength ammonium sulphide solution. The maintenance of pregnancy rate was calculated as the quotient of the number of living foetuses and the total number of nidation sites (both resorbed and dead foetuses and nidation sites). For certain test substances, the pregnancy-maintaining doses (ED50) indicated in Table 1 were determined. For drospirenone, this value is 3.5 mg/kg/day.

The derivatives according to the invention having the general chemical formula have a very strong gestagenic activity. It was moreover found that the derivatives according to the invention show antimineralcorticoid action in vitro. They should therefore have in vivo potassium-retaining, natriuretic (antimeralcorticoid) action. These properties were determined using the test described below:

For the culturing of the cells used for the assay, the culture medium used was DMEM (Dulbecco's Modified Eagle Medium: 4500 mg/ml of glucose; PAA, #E15-009) with 10% FCS (Biochrom, S0115, batch #615B), 4 mM L-glutamine, 1% penicillin/streptomycin, 1 mg/ml of G418 and 0.5 μg/ml of puromycin.

Reporter cell lines were grown in a density of 4×104 cells per hollow in white, nontransparent tissue culture plates in each case having 96 hollows (PerkinElmer, #P12-106-017) and kept in 6% DCC-FCS (activated carbon-treated serum, for the removal of interfering components contained in the serum). The compounds to be investigated were added eight days later, and the cells were incubated with the compounds for 16 hours. The experiments were carried out in triplicate. At the end of the incubation, the effector-containing medium was removed and replaced by lysis buffer. After luciferase assay substrate (promega, #E1501) had been added, the plates containing the 96 hollows were then introduced into a microplate luminometer (Pherastar, BMG labtech), and the luminescence was measured. The 1050 values were evaluated using software for the calculation of dose-activity relationships. Experimental results are presented in Table 1:

TABLE 1

| | | MR Antagonism activity | |
|---|---|---|---|
| | MR Antagonism IC50 | [% of the maximum | PR in vivo ED50 |
| Compound | [nM] | effect] | [mg/kg/d s.c.] |
| 6β,7β; 15β,16β-Bismethylene-17β- | 4.7 | 98.06 | 7.3 |
| cyanoandrost-4-en-3-one | | | |
| 17β-Cyano-6,6-ethanediylandrost-4- | 20.0 | 99.16 | 25.0 |
| en-3-one | | | |
| 17α-Allyl-17β-cyanoandrost-4-en-3- | 990.0 | 65.30 | |
| one | | | |
| 17β-Cyano-17α-methyl-15β,16β- | 42.0 | 98.30 | 10.0 |
| methyleneandrost-4-en-3-one | | | |

The following examples for the synthesis of preferred invention serve for the further illustration of the invention. The new intermediates disclosed in the individual synthesis examples are, just like the 17β-cyano-19-androst-4-ene derivatives according to the invention, essential to the invention.

Many of the reactions described below lead to epimer mixtures. Usually, the chromatographic separation of these mixtures via preparative HPLC is carried out under the following conditions: separations were carried out on a chiral normal phase, the stationary phase usually used being Chiralpak AD-H 5μ. Customarily, elution was carried out using a mixture of hexane and ethanol. In some cases, however, other eluent mixtures, such as, for example, mixtures of methanol and ethanol, were used:

### EXAMPLE 1

### 17β-Cyano-15β,16β-methyleneandrost-4-en-3-one

### 1a)

### 3-Methoxy-15β,16β-methyleneandrost-3(4),5(6)-dien-17-one

50 g of 15β,16β-methyleneandrost-4-ene-3,17-dione were dissolved in 1 l of methanol and 175 ml of trimethyl orthoformate. With stirring at 25° C., 250 mg of p-toluenesulphonic acid were added. After a short time, the product precipitated out. The mixture was stirred at 25° C. for 1 hour and at −5° C. for 1 hour. The mixture was neutralized with pyridine and filtered with suction to obtain 3-methoxy-15β,16β-methyleneandrost-3(4),5(6)-dien-17-one (48 g).

¹H NMR (300 MHz, CDCl₃, TMS as an internal standard, selected signals): δ=3.54 (s, O—CH₃), 5.12 (m, 4-H), 5.25 (m, 6-H).

MS (Cl+) m/z (rel. intensity)=312 (100); corresponds to C₂₁H₂₈O₂.

### 1b)

### 17β-Cyano-3-methoxy-15β,16β-methyleneandrostane-3(4),5(6)-diene

The solution of 23.43 g of TOSMIC® in 140 ml of dimethoxyethane was added slowly with ice cooling over a period of 1.5 hours to a solution of 25 g of 15β,16β-methylene-3-methoxyandrostane-3(4),5(6)-dien-17-one and 45 g of potassium tert-butoxide in 1 l of dimethoxyethane and 300 ml of tert-butanol, and the mixture was then left to stir at room temperature for another 3 hours. The reaction mixture was poured onto ice-cold semisaturated sodium chloride solution, and the precipitated product was filtered off with suction, washed with water and dried overnight in a vacuum drying cabinet (50° C., 200 mbar). 17β-Cyano-3-methoxy-15β,16β-methyleneandrostane-3(4),5)(6)-diene (23.7 g) was obtained in the form of beige crystals.

¹H NMR (300 MHz, CDCl₃, TMS as an internal standard, selected signals): δ=0.42 [m, 15′-H(β)], 2.73 [d, J=4.5 Hz, 17-H(α)], 5.14 (br. s, 4-H), 5.27 (m, 6-H).

MS (Cl+) m/z (rel. intensity)=324 (100), 341 (85); corresponds to C₂₂H₂₉NO.

### 1c)

### 17β-Cyano-15β,16β-methyleneandrost-4-en-3-one

5 ml of sulphuric acid (8% by weight) were added at room temperature to a solution of 700 mg of 17β-cyano-3-methoxy-15β,16β-methyleneandrostane-3(4),5(6)-diene in 10 ml of methanol, and the mixture was left to stir at this temperature for 2 hours. After the reaction had been terminated with saturated bicarbonate solution, the mixture was extracted with dichloromethane, washed with H₂O and saturated sodium chloride solution, dried over sodium sulphate and concentrated on a rotary evaporator. In the course of this, 17β-cyano-15β,16β-methyleneandrost-4-en-3-one (599 mg) crystallized out.

¹H NMR (300 MHz, CDCl₃, TMS as an internal standard, selected signals): δ=0.44 [m, 15′-H(β)], 2.74 [d, J=4.5 Hz, 17-H(α)], 5.44 (br. s, 4-H).

MS (EI+) m/z (rel. intensity)=309 (50); corresponds to C₂₁H₂₇NO.

### EXAMPLE 2

### 17β-Cyano-6β-hydroxymethyl-15β,16β-methyleneandrost-4-en-3-one

9.5 g of 17β-cyano-15β,16β-methyleneandrost-4-en-3-one were taken up in 60 ml of methanol, admixed with 4.8 ml of pyrrolidine and heated at reflux for 1 hour. After cooling, the solid was filtered off with suction, washed with a little cold methanol and suction-dried. The crystals (11 g) were dissolved in 135 ml of toluene and 235 ml of ethanol; 11.5 ml of 30% formaldehyde solution were added. After stirring at room temperature for 2 hours, the mixture was concentrated to dryness and chromatographed on silica gel. 17β-Cyano-6β-hydroxymethyl-15β,16β-methyleneandrost-4-en-3-one (4.7 g) was obtained.

¹H NMR (300 MHz, CDCl₃, TMS as an internal standard, selected signals): δ=0.47 [m, 15′-H(β)], 2.76 [d, J=4.5 Hz, 17-H(α)], AB signal (δ_{A}=3.68, δ_{B}=3.80, J_{AB}=10.5 Hz additionally split by J_{H(A),6-H}=7.4 Hz, J_{H(B),6-H}=10.5 Hz), 5.84 (s, 4-H).

MS (EI+) m/z (rel. intensity)=339 (37); corresponds to C₂₂H₂₉NO₂.

### EXAMPLE 3

### 17β-Cyano-6,6-ethylene-15β,16β-methyleneandrost-4-en-3-one

### 3a)

### 17β-Cyano-15β,16β-methylene-6β-tosyloxymethylandrost-4-en-3-one

2.93 g of toluenesulphonyl chloride were added in one portion to a solution of 1.74 g of 17β-cyano-6β-hydroxymethyl-15β,16β-methyleneandrost-4-en-3-one in 20 ml of pyridine, and the mixture was left to stir at room temperature for 6 hours. Thereafter, the reaction mixture was added to ice-cold 1 N hydrochloric acid, and the precipitated crude product was filtered off with suction and dissolved again in ethyl acetate. After washing twice each with water, saturated bicarbonate solution and saturated sodium chloride solution and drying the organic phase with sodium sulphate, after concentration to dryness, 17β-cyano-15β,16β-methylene-6β-tosyloxymethylandrost-4-en-3-one was obtained, and was used immediately in the next stage.

¹H NMR (300 MHz, CDCl₃, TMS as an internal standard, selected signals): δ=0.46 [m, 15′-H(β)], AB signal (δ_{A}=3.95, δ_{B}=4.20, J_{AB}=9.5 Hz additionally split by J_{H(A),6-H}=7.0 Hz, J_{H(B),6-H}=9.5 Hz), 5.72 (s, 4-H).

### 3b)

### 17β-Cyano-6,6-ethylene-15β,16β-methyleneandrost-4-en-3-one

913 mg of sodium hydride were added in portions at room temperature to a solution of 6.02 g of trimethylsulphoxonium iodide in 50 ml of dry DMSO, and, after the addition had ended, the mixture was left to stir at room temperature for 1 hour. Subsequently, the solution of 3.13 g of 17β-cyano-15β,16β-methylene-6β-tosyloxymethylandrost-4-en-3-one was added to the ylide formed and stirred at room temperature for a further 6 hours. After the termination of the reaction by adding 350 ml of water, extraction twice with 150 ml of ethyl acetate, washing of the organic phase with water and saturated sodium chloride solution and drying over sodium sulphate, the organic phase was treated at room temperature with activated carbon for 15 minutes. After filtration through a layer of Celite®, 17β-cyano-6,6-ethylene-15β,16β-methyleneandrost-4-en-3-one crystallized out on concentration of the organic phase (K1 503 mg, K2 379 mg).

¹H NMR (300 MHz, CDCl₃, TMS as an internal standard, selected signals): δ=0.38-0.53 [m, (3H) spiroethylene], 0.88 [m, (1H) spiroethylene], 2.75 [d, J=4.5 Hz, 17-H(α)], 5.65 (s, 4-H).

MS (EI+) m/z (rel. intensity)=335 (100); corresponds to C₂₃H₂₈NO.

### EXAMPLE 4

### 17β-Cyano-6-exo-methylene-15β,16β-methyleneandrost-4-en-3-one

1 ml of 6 N hydrochloric acid were added at room temperature to a solution of 1 g of 17β-cyano-6β-hydroxymethyl-15β,16β-methyleneandrost-4-en-3-one in 10 ml of dioxane, and the mixture was left at this temperature for 2 hours. Subsequently, the reaction mixture was added to 250 ml of ice-cold semisaturated bicarbonate solution and extracted 2× with 150 ml of ethyl acetate. After treating the combined organic phases with sodium sulphate and activated carbon, they were filtered through a Celite® layer and concentrated to dryness. Flash chromatography on silica gel [hexane/ethyl acetate (0-25%)] afforded 17β-cyano-6-exo-methylene-15β,16β-methyleneandrost-4-en-3-one (399 mg).

¹H NMR (300 MHz, CDCl₃, TMS as an internal standard, selected signals): δ=0.48 [m, 15′-H(β)], 2.77 [d, J=4.7 Hz, 17-H(α)], 5.02 and 5.12 (dd→t, in each case J=2.0 Hz, ═CH₂), 5.94 (d, J=0.6 Hz, 4-H).

MS (EI+) m/z (rel. intensity)=321 (96); corresponds to C₂₂H₂₇NO.

### EXAMPLE 5

### 17β-Cyano-6α-methyl-15β,16β-methyleneandrost-4-en-3-one

300 mg of Wilkinson's catalyst were added under an argon atmosphere to a solution of 330 mg of 17β-cyano-6-exo-methylene-15β,16β-methyleneandrost-4-en-3-one in 40 ml of toluene and 10 ml of ethanol, and hydrogenated under standard hydrogen pressure by means of a shaking apparatus for 3 hours. After the removal of the catalyst by flash chromatography on silica gel [hexane/ethyl acetate (0-50%)], the mixture of the 6-epimers was obtained in a 17β-cyano-6β-methyl-15β,16β-methyleneandrost-4-en-3-one:17β-cyano-6α-methyl-15β,16β-methyleneandrost-4-en-3-one ratio=2.5:1. Acidic epimerization in dichloromethane with catalytic amounts of p-toluenesulphonic acid and another flash chromatography on silica gel [hexane/ethyl acetate (0-50%)] afforded pure 17β-cyano-6α-methyl-15β,16β-methyleneandrost-4-en-3-one (39 mg).

¹H NMR (300 MHz, CDCl₃ TMS as an internal standard, selected signals): δ=0.46 [m, 15′-H(β)], 1.12 (d, J=6.3 Hz, 6-CH₃), 2.75 (d, J=4.6 Hz, 17-H(α)], 5.82 (d, J=1.3 Hz, 4-H).

MS (EI+) m/z (rel. intensity)=324 (95), 341 (55); corresponds to C₂₂H₂₉NO.

### EXAMPLE 6

### 17β-Cyano-15β,16β-methyleneandrosta-4,6-dien-3-one

A suspension of 3.4 g of 17β-cyano-3-methoxy-15β,16β-methyleneandrost-3(4),5(6)-diene in 100 ml of 1-methyl-2-pyrrolidone was admixed successively at 0° C. with 4 ml of a 10% sodium acetate solution and, at this temperature, with 1.6 g of 1,3-dibromo-5,5-dimethylhydantoin in portions, stirred at 0° C. (ice bath) for 0.5 hour, admixed with 1.5 g of lithium bromide and 1.3 g of lithium carbonate, and stirred at bath temperature 100° C. for 3.5 hours. Subsequently, the mixture was stirred into ice-water/sodium chloride and the precipitate was filtered off. 17β-Cyano-15β,16β-methyleneandrosta-4,6-dien-3-one (2.42 g) was obtained.

¹H NMR (300 MHz, CDCl₃, TMS as an internal standard, selected signals): δ=0.53 [m, 15′-H(β)], 2.78 [d, J=4.5 Hz, 17-H(α)], 5.70 (s, 4-H), 6.18 (dd, J=2.8 Hz, J=9.8 Hz, 5-H*), 6.33 (dd, J=2.1 Hz, J=9.8 Hz, 6-H*), *=assignment interchangeable.

### EXAMPLE 7

### 6β,7β-15β,16β-Bismethylene-17β-cyanoandrost-4-en-3-one and 6α,7α-15β,16β-bismethylene-17β-cyanoandrost-4-en-3-one

468 mg of sodium hydride were added in portions at room temperature to a solution of 3.09 g of trimethylsulphoxonium iodide in 25 ml of dry dimethyl sulphoxide and, after the addition had ended, stirred at room temperature for 1 hour. Subsequently, the solution of 1.0 g of 17β-cyano-15β,16β-methyleneandrosta-4,6-dien-3-one was added to the ylide formed and the mixture was stirred at room temperature for a further 6 hours. After the reaction had been terminated by adding 150 ml of ammonium chloride solution, extracting twice with 75 ml of ethyl acetate, washing the organic phase with water and saturated sodium chloride solution, and drying over sodium sulphate, the organic phase was concentrated to dryness. Flash chromatography twice on silica gel [hexane/ethyl acetate (0-50%)] afforded, as the less polar fraction, 6α,7α-15β,16β-bismethylene-17β-cyanoandrost-4-en-3-one (59 mg) and, as the more polar fraction, 6β,7β-15β,16β-bismethylene-17β-cyanoandrost-4-en-3-one (67 mg).

### Fraction 1: 6α,7α-15β,16β-bismethylene-17β-cyanoandrost-4-en-3-one ¹H NMR (300 MHz, CDCl₃, TMS as an internal standard, selected signals): δ=0.53, 0.63, 0.87 and 0.98 [4×m, (each 1H) cyclopropyl], 2.80 [d, J=4.3 Hz, 17-H(α)], 6.01 (s, 4-H).

MS (Cl+) m/z (rel. intensity)=322 (100), 339 (33); corresponds to C₂₂H₂₇NO.

### Fraction 2: 6β,7β-15β,16β-bismethylene-17β-cyanoandrost-4-en-3-one

¹H NMR (300 MHz, CDCl₃, TMS as an internal standard, selected signals): δ=0.57 and 0.92 [2×m, (each 1H) cyclopropyl], 2.84 [d, J=4.3 Hz, 17-H(α)], 6.07 (s, 4-H).

MS (EI+) m/z (rel. intensity)=322 (100), 339 (33); corresponds to C₂₂H₂₇NO.

### EXAMPLE 7

### Variant 2

### 6β,7β-15β,16β-Bismethylene-17β-cyanoandrost-4-en-3-one

### 7-variant 2-a)

### 6β,7β-15β,16β-Bismethylene-3β-5β-bishydroxy-17β-cyanoandrostane and 6β,7β-15β,16β-bismethylene-3β-5β-bishydroxy-17α-cyanoandrostane

6β,7β-15β,16β-Bismethylene-3β,5β-dihydroxyandrostan-17-one (Angew. Chemie 1982, 94, 718-719) was converted analogously to the method specified in Example 1b. Chromatography on silica gel with a mixture of hexane and ethyl acetate afforded 6β,7β-15β,16β-bismethylene-3β-5β-bishydroxy-17β-cyanoandrostane and 6β,7β-15β,16β-bismethylene-3β-5β-bishydroxy-17α-cyanoandrostane.

### 6β,7β-15β,16β-Bismethylene-3β-5β-bishydroxy-17β-cyanoandrostane

¹H NMR (D6-DMSO): 0.41 (m, 2H), 0.61 (m, 1H), 0.73 (s, 3H), 0.83 (s, 3H), 2.97 (s broad, 1H), 3.79 (s broad, 1H), 4.31 (s broad, 1H), 4.79 (s broad, 1H)

### 6β,7β-15β,16β-Bismethylene-3β-5β-bishydroxy-17α-cyanoandrostane

¹H NMR (D6-DMSO): 0.41 (m, 2H), 0.61 (m, 1H), 0.73 (s, 3H), 0.80 (s, 3H), 3.05 (s broad, 1H)

### 7-Variant 2-b)

### 6β,7β-15β,16β-Bismethylene-17β-cyanoandrost-4-en-3-one

6β,7β-15β,16β-Bismethylene-3β-5β-bishydroxy-17β-cyanoandrostane was converted analogously to the method specified in Example 30e. 6β,7β-15β,16β-Bismethylene-17β-cyanoandrost-4-en-3-one was obtained.

The NMR data are identical to those reported in Example 7.

### EXAMPLE 8

### 17β-Cyano-7α-methyl-15β,16β-methyleneandrost-4-en-3-one

67 mg of copper(I) chloride were added at room temperature to a solution of 1.0 g of 17β-cyano-15β,16β-methyleneandrostane-4,6-dien-3-one in 50 ml of tetrahydrofuran, and the mixture was stirred for 10 minutes, before it was cooled to −15° C., admixed with 450 mg of aluminium chloride, stirred at this temperature for 30 minutes, admixed dropwise with 4.5 ml of methylmagnesium bromide solution (3 M in tetrahydrofuran) and stirred at −15° C. for one hour. For workup, the reaction mixture was admixed at −15° C. with 30 ml of 2 M hydrochloric acid, stirred at room temperature for 0.5 hour, added to water, extracted three times with ethyl acetate, dried over sodium sulphate, concentrated under reduced pressure and chromatographed on silica gel with hexane/ethyl acetate (0-50%). 17β-Cyano-7α-methyl-15β,16β-methyleneandrost-4-en-3-one (149 mg) was obtained.

¹H NMR (300 MHz, CDCl₃, TMS as an internal standard, selected signals): δ=0.45 [m, 15′-H(β)], 0.88 (d, J=7.1 Hz, 7-Me), 1.08 and 1.21 [2×s, in each case (3H), 2×Me], 2.75 [d, J=4.6 Hz, 17-H(α)], 5.76 (s, 4-H).

MS (Cl+) m/z (rel. intensity)=324 (100), 341 (55); corresponds to C₂₂H₂₉NO.

### EXAMPLE 9

### 17β-Cyano-17α-methyl-15β,16β-methyleneandrost-4-en-3-one

### 9a)

### 17β-Cyano-3-methoxy-17α-methyl-15β,16β-methyleneandrostane-3(4),5(6)-diene

At −78° C., a cold fresh solution of lithium diisopropylamide (LDA), which had been prepared beforehand from 12.1 ml of diisopropylamine and 54.1 ml of n-BuLi (1.6 M in hexane) in 82 ml of tetrahydrofuran at 0° C., was added to a solution of 8 g of 17-cyano-3-methoxy-15β,16β-methyleneandrostane-3(4),5(6)-diene, and the mixture was left at −78° C. for 1 hour. Before the addition of 6.9 ml of methyl iodide which then followed, the mixture was cooled further to −90° C. After the addition had ended, the reaction mixture was allowed to warm slowly to room temperature overnight. The reaction was terminated by the addition of saturated ammonium chloride solution, extracted with ethyl acetate and washed with water and saturated sodium chloride solution. Drying of the organic phase with sodium sulphate, concentrating to dryness and flash chromatography on silica gel [hexane/ethyl acetate (0-30%)] afforded 17β-cyano-3-methoxy-17α-methyl-15β,16β-methyleneandrostane-3(4),5(6)-diene (6.5 g).

¹H NMR (300 MHz, CDCl₃, TMS as an internal standard, selected signals): δ=0.40 [m, 15′-H(β)], 1.01 (s, Me), 1.15 (s, Me), 1.38 (s, Me), 3.58 (s, O—CH₃), 5.15 (m, 4-H), 5.27 (m, 6-H).

MS (Cl+) m/z (rel. intensity)=355 (100), 338 (53); corresponds to C₂₃H₃₁NO.

### 9b)

### 17β-Cyano-17α-methyl-15β,16β-methyleneandrost-4-en-3-one

According to the method of Example 1c, 385 mg of 17β-cyano-3-methoxy-17α-methyl-15β,16β-methyleneandrostane-3(4),5(6)-diene, after crystallization from ethyl acetate, afforded 17β-cyano-17α-methyl-15β,16β-methyleneandrost-4-en-3-one (285 mg).

¹H NMR (300 MHz, CDCl₃, TMS as an internal standard, selected signals): δ=0.43 [m, 15′-H(β)], 1.16; 1.21 and 1.38 [3×s, in each case (3H), 3×Me], 5.75 (d, J=1.1 Hz, 4-H).

MS (Cl+) m/z (rel. intensity)=324 (38), 341 (100); corresponds to C₂₂H₂₉NO.

### EXAMPLE 10

### 17α-Allyl-17β-cyano-15β,16β-methyleneandrost-4-en-3-one

### 10a)

### 17α-Allyl-17β-cyano-3-methoxy-15β,16β-methyleneandrostane-3(4),5(6)-diene

According to the method of Example 9a, 1 g of 17-cyano-3-methoxy-15β,16β-methyleneandrostane-3(4),5(6)-diene, with allyl bromide as the alkylating agent, after flash chromatography, afforded 17α-allyl-17β-cyano-3-methoxy-15β,16β-methyleneandrostane-3(4),5(6)-diene (358 mg).

¹H NMR (300 MHz, CDCl₃, TMS as an internal standard, selected signals): δ=0.41 [m, 15′-H(β)], 3.58 (s, O—CH₃), 5.15 (m, 4-H), 5.25 [m, (3H), 6-H and ═CH₂], 6.05 [m, (1H), —CH═].

### 10b)

### 17α-Allyl-17β-cyano-15β,16β-methyleneandrost-4-en-3-one

According to the method of Example 1c, 300 mg of 17α-allyl-17β-cyano-3-methoxy-15β,16β-methyleneandrostane-3(4),5(6)-diene, after flash chromatography, afforded 17α-allyl-17β-cyano-15β,16β-methyleneandrost-4-en-3-one (210 mg).

¹H NMR (300 MHz, CDCl₃, TMS as an internal standard, selected signals): δ=0.45 [m, 15′-H(β)], 5.18-5.30 [m, (2H), ═CH₂], 5.76 (d, J=1.7 Hz, 4-H), 6.03 [m, (1H), —CH═].

MS (Cl+) m/z (rel. intensity)=350 (100), 367 (68); corresponds to C₂₄H₃₁NO.

### EXAMPLE 11

### 17β-Cyano-6β-hydroxymethyl-17α-methyl-15β,16β-methyleneandrost-4-en-3-one

According to the method of Example 2, 5.90 g of 17β-cyano-17α-methyl-15β,16β-methyleneandrost-4-en-3-one, after crystallization from ethyl acetate and flash chromatography of the mother liquors on silica gel [hexane/ethyl acetate (0-50%)], afforded 17β-cyano-6β-hydroxymethyl-17α-methyl-15β,16β-methyleneandrost-4-en-3-one (2.22 g).

¹H NMR (300 MHz, CDCl₃, TMS as an internal standard, selected signals): δ=0.50 [m, 15′-H(β)], 1.21; 1.27 and 1.43 [3×s, in each case (3H), 3×Me], AB signal (δ_{A}=3.73, δ_{B}=3.85, J_{AB} approx. 10.0 Hz broad signals, additionally split by J_{H(A),6-H}=7.5 Hz, J_{H(b),6-H} approx. 10.0 Hz), 5.89 (s, 4-H).

MS (Cl+) m/z (rel, intensity)=341 (100), 354 (35), 371 (22); corresponds to C₂₃H₃ᵢNO₂.

### EXAMPLE 12

### 17β-Cyano-15α,16α-methyleneandrost-4-en-3-one

### 12a)

### 17β-Cyano-3β-hydroxy-15α,16α-methyleneandrost-5(6)-ene

According to the method of Example 1b, 24.2 g of 3β-acetoxy-15α,16α-methyleneandrost-5(6)-ene, after flash chromatography on silica gel [hexane/ethyl acetate (0-50%)] and fractional crystallization from ethyl acetate, afforded 17β-cyano-3β-hydroxy-15α,16α-methyleneandrost-5(6)-ene (3.2 g) and 17α-cyano-3β-hydroxy-15α,16α-methyleneandrost-5(6)-ene (3.6 g).

### 17β-Cyano-3β-hydroxy-15α,16α-methyleneandrost-5(6)-ene

¹H NMR (300 MHz, CDCl₃, TMS as an internal standard, selected signals): δ=0.54 [m, (2H), cyclopropyl], 0.90 [m, (1H), cyclopropyl], 1.03 and 1.17 [2×s, in each case (3H), 2×Me], 3.52 (m, 3-H), 5.37 (m, 6-H).

MS (EI+) m/z (rel. intensity)=311 (88); corresponds to C₂₁H₂₉NO.

### 17α-Cyano-3β-hydroxy-15α,16α-methyleneandrost-5(6)-ene

¹H NMR (300 MHz, CDCl₃, TMS as an internal standard, selected signals): δ=0.78 [m, (1H), cyclopropyl], 0.89-0.99 [m, (2H), cyclopropyl], 1.02 and 1.08 [2×s, in each case (3H), 2×Me], 2.79 [d, J=7.7 Hz, 17-H (β)], 3.54 (m, 3-H), 5.38 (m, 6-H).

MS (EI+) m/z (rel. intensity)=311 (18); corresponds to C₂₁H₂₉NO.

### 12b)

### 17β-Cyano-15α,16α-methyleneandrost-4-en-3-one

2.10 g of aluminium triisopropoxide were added in one portion to the solution of 3.2 g of 17β-cyano-3β-hydroxy-15α,16α-methyleneandrost-5(6)-ene in 80 ml of 2-butanone, and the mixture was heated to boiling for 15 hours. Subsequently, the reaction terminated by adding saturated ammonium chloride solution and extracted 3 times with ethyl acetate, and the organic phase was washed with saturated sodium chloride solution and dried over sodium sulphate. After concentration, flash chromatography on silica gel [hexane/ethyl acetate (0-50%)] afforded 17β-cyano-15α,16α-methyleneandrost-4-en-3-one (3.0 g).

¹H NMR (300 MHz, CDCl₃, TMS as an internal standard, selected signals): δ=0.55 [m, (2H), cyclopropyl], 0.81-0.97 [m, (3H)], 1.21 [s, (6H), 2×Me], 5.74 (br. s, 4-H).

MS (Cl+) m/z (rel. intensity)=310 (100), 327 (23); corresponds to C₂₁H₂₇NO.

### EXAMPLE 13

### 17β-Cyano-6β-hydroxymethyl-15α,16α-methyleneandrost-4-en-3-one

According to the method of Example 2, 3.0 g of 17β-cyano-15α,16α-methyleneandrost-4-en-3-one, after flash chromatography on silica gel [hexane/ethyl acetate (0-50%)], afforded 17β-cyano-6β-hydroxymethyl-15α,16α-methyleneandrost-4-en-3-one (850 mg).

¹H NMR (300 MHz, CDCl₃, TMS as an internal standard, selected signals): δ=0.56 [m, (2H), cyclopropyl], 0.91 [m, (1H), cyclopropyl], 1.22 [s, (6H), 2×Me], AB signal (δ_{A}=3.68, δ_{B}=3.77, highly broadened signals), 5.83 (s, 4-H).

MS (Cl+) m/z (rel. intensity)=340 (100), 357 (51); corresponds to C₂₂H₂₉NO₂.

### EXAMPLE 14

### 17β-Cyano-15α,16α-methylene-6β-tosyloxymethylandrost-4-en-3-one and 17β-cyano-6-exo-methylene-15α,16α-methyleneandrost-4-en-3-one

According to the method of Example 3a, 700 mg of 17β-cyano-6β-hydroxymethyl-15α,16α-methyleneandrost-4-en-3-one, after flash chromatography on silica gel [hexane/ethyl acetate (0-50%)], afforded 17β-cyano-15α,16α-methylene-6β-tosyloxymethylandrost-4-en-3-one (880 mg) and, in its first runnings as a minor component, 17β-cyano-6-exo-methylene-15α,16α-methyleneandrost-4-en-3-one (22 mg).

### Fraction 1: 17β-Cyano-6-exo-methylene-15α,16α-methyleneandrost-4-en-3-one

¹H NMR (300 MHz, CDCl₃, TMS as an internal standard, selected signals): δ=0.64 [m, (2H), cyclopropyl], 1.09 [m, (1H), cyclopropyl], 1.16 and 1.25 [2×s, in each case (3H), 2×Me], 5.04 and 5.15 (dd→1, in each case J=1.9 Hz, ═CH₂), 5.97 (s, 4-H).

MS (Cl+) m/z (rel. intensity)=322 (100), 339 (28); corresponds to C₂₂H₂₇NO.

### Fraction 2: 17β-Cyano-15α,16α-methylene-6β-tosyloxymethylandrost-4-en-3-one

¹H NMR (300 MHz, CDCl₃, TMS as an internal standard, selected signals): δ=0.56 [m, (2H), cyclopropyl], 0.91 [m, (1H), cyclopropyl], 1.13 and 1.22 [2×s, in each case (3H), 2×Me], 2.50 [s, (3H), C₆H₄—CH₃], AB signal (δ_{A}=3.95, δ₅=4.29, J_{AB}=9.7 Hz additionally split by J_{H(A),6-H}=6.2 Hz, J_{H(B),6-H}=9.7 Hz), 5.77 (s, 4-H), AA′BB′ signal [δ_{A}=7.40, δ₅=7.82, in each case (2H), C₆H₄].

MS (Cl+) m/z (rel. intensity)=494 (5), 511 (15); corresponds to C₂₉H₃₅NO₄S.

### EXAMPLE 15

### 17β-Cyano-6,6-ethylene-15α,16α-methyleneandrost-4-en-3-one

According to the method of Example 3b, 860 mg of 17β-cyano-15α,16α-methylene-6β-tosyloxymethylandrost-4-en-3-one, after flash chromatography on silica gel [hexane/ethyl acetate (0-50%)], afforded 17β-cyano-6,6-ethylene-15α,16α-methyleneandrost-4-en-3-one (265 mg).

¹H NMR (300 MHz, CDCl₃, TMS as an internal standard, selected signals): δ=0.45-0.63 [m, (4H) spiroethylene and cyclopropyl], 0.88 [m, (1H) spiroethylene], 1.02 [m, (1H), cyclopropyl], 1.28 and 1.32 [2×s, in each case (3H), 2×Me], 5.69 (s, 4-H).

MS (EI+) m/z (rel. intensity)=335 (88); corresponds to C₂₃H₂₉NO.

### EXAMPLE 16

17β-Cyano-17α-methyl-15α,16α-methyleneandrost-4-en-3-one

### 16a)

### 17α-Cyano-15α,16α-methylene-3β-tri isopropylsilyloxyandrost-5(6)-ene

The solution of 5.44 ml of triisopropylsilyl chloride in 2.5 ml of tetrahydrofuran was added slowly at 0° C. to a solution of 3.6 g of 17α-cyano-3β-hydroxy-15α,16α-methyleneandrost-5(6)-ene, 1.7 g of imidazole and 141 mg of dimethylaminopyridine in 20 ml of dimethylformamide (DMF). Subsequently, the mixture was allowed to warm to room temperature overnight, poured onto saturated bicarbonate solution and extracted with ethyl acetate. The organic phase was washed 5× with water and finally with saturated sodium chloride solution, and concentrated to dryness. The crude 17α-cyano-15α,16α-methylene-3β-triisopropylsilyloxyandrost-5(6)-ene product (7.3 g) was used directly in the next stage.

¹H NMR (300 MHz, CDCl₃, TMS as an internal standard, selected signals): δ=0.78 [m, (1H), cyclopropyl], 0.88-0.99 [m, (2H), cyclopropyl], 1.02 and 1.08 [2×s, in each case (3H), 2×Me, overlapped by δ=1.05, br. s, (18H), TiPS-Me], 2.79 [d, J=7.1 Hz, 17-H (β)], 3.56 (m, 3-H), 5.33 (d, J=4.8 Hz, 6-H).

MS (EI+) m/z (rel. intensity)=468 (12); corresponds to C₃₀H₄₉NOSi.

### 16b)

### 17β-Cyano-17α-methyl-15α,16α-methylene-3β-triisopropylsilyloxyandrost-5(6)-ene in a mixture with 17α-cyano-17β-methyl-15α,16α-methylene-3β-triisopropylsilyloxyandrost-5(6)-ene

According to the method of Example 9a, 5.29 g of 17α-cyano-15α,16α-methylene-3β-triisopropylsilyloxyandrost-5(6)-ene, with Me-I as the alkylating agent, after flash chromatography, afforded the mixture of the 17-epimers of 17-cyano-17-methyl-15α,16α-methylene-3β-triisopropylsilyloxyandrost-5(6)-ene (3.65 g).

¹H NMR (300 MHz, CDCl₃, TMS as an internal standard, selected signals): δ=0.60-0.72 [m, (1H), cyclopropyl], 1.05, [br. s, (18H), TiPS-Me], 3.54 (m, 3-H), 5.33 (m, 6-H).

MS (Cl+) m/z (rel. intensity)=499 (55); corresponds to C₃₁H₅₁NOSi.

### 16c)

### 17β-Cyano-3β-hydroxy-17α-methyl-15α,16α-methyleneandrost-5(6)-ene in a mixture with 17α-cyano-3β-hydroxy-17β-methyl-15α,16α-methyleneandrost-5(6)-ene

9 ml of tetrabutylammonium fluoride (TBAF) (1 M in tetrahydrofuran) were added at room temperature to a solution of 3.6 g of the 17-epimers of 17-cyano-17-methyl-15α,16α-methylene-3β-triisopropylsilyloxyandrost-5(6)-ene in 5 ml of tetrahydrofuran, and the mixture was left to stir for a further 4 hours. After the reaction had been terminated by adding saturated bicarbonate solution, extracting with ethyl acetate, washing the organic phase with water and saturated sodium chloride solution, drying with sodium sulphate and concentrating to dryness, subsequent flash chromatography afforded the 17-epimer mixture of 17-cyano-3β-hydroxy-17-methyl-15α,16α-methyleneandrost-5(6)-ene (1.9 g).

¹H NMR (300 MHz, CDCl₃, TMS as an internal standard, selected signals): δ=0.74 [m, (1H), cyclopropyl], 3.57 (m, 3-H), 5.42 (m, 6-H).

### 16d)

### 17β-Cyano-17α-methyl-15α,16α-methyleneandrost-4-en-3-one

According to the method of Example 12b, 1.9 g of the 17-epimers of 17-cyano-3β-hydroxy-17-methyl-15α,16α-methyleneandrost-5(6)-ene, after preparative HPLC chromatography, afforded 17β-cyano-17α-methyl-15α,16α-methyleneandrost-4-en-3-one (335 mg).

¹H NMR (300 MHz, CDCl₃, TMS as an internal standard, selected signals): δ=0.69 [m, (2H), cyclopropyl], 0.82 [m, (1H), cyclopropyl], 0.96 [m, (1H), cyclopropyl], 1.14; 1.21 and 1.33 [3×s, in each case (3H), 3×Me], 5.74 (s, 4-H).

MS (EI+) m/z (rel. intensity)=323 (100); corresponds to C₂₂H₂₉NO.

### EXAMPLE 17

### 17β-Cyano-15α,16α-methyleneandrost-4,6-dien-3-one

### 17a)

### 17β-Cyano-3-methoxy-15α,16α-methyleneandrost-3(4),5(6)-dien-17-one in a mixture with 17α-cyano-3-methoxy-15α,16α-methyleneandrost-3(4),5(6)-dien-17-one

According to the method of Example 1a, 7 g of the 17-epimers of 17-cyano-15α,16α-methyleneandrost-4-en-3-one, after workup, afforded the 17-epimers of 17-cyano-3-methoxy-15α,16α-methyleneandrost-3(4),5(6)-diene (7.6 g), which were used directly in the next stage.

### 17b)

### 17β-Cyano-15α,16α-methyleneandrosta-4,6-dien-3-one

According to the method of Example 6, 7.6 g of the 17-epimers of 17-cyano-3-methoxy-15α,16α-methyleneandrost-3(4),5(6)-diene, after preparative HPLC chromatography of a portion of the resulting crude product, afforded 17β-cyano-15α,16α-methyleneandrosta-4,6-dien-3-one (48 mg).

¹H NMR (300 MHz, CDCl₃, TMS as an internal standard, selected signals): δ=0.66 [m, (1H), cyclopropyl], 0.78 [m, cyclopropyl], 1.19 and 1.32 [2×s, in each case (3H), 2×Me], 5.74 (s, 4-H), 6.21 (dd, J=2.8 Hz, J=9.8 Hz, 5-H*), 6.34 (dd, J=1.9 Hz, J=10.0 Hz, 6-H*), *=assignment interchangeable.

MS (EI+) m/z (rel. intensity)=307 (26); corresponds to C₂₁H₂₅NO.

### EXAMPLE 18

### 17β-Cyano-7α-methyl-15α,16α-methyleneandrost-4-en-3-one

According to the method of Example 8, 2.2 g of the 17-epimers of 17-cyano-15α,16α-methyleneandrosta-4,6-dien-3-one, after preparative HPLC chromatography, afforded 17β-cyano-7α-methyl-15α,16α-methyleneandrost-4-en-3-one (257 mg).

¹H NMR (300 MHz, CDCl₃, TMS as an internal standard, selected signals): δ=0.58 [m, (1H), cyclopropyl], 0.69 [m, (1H), cyclopropyl], 0.86 (d, J=7.2 Hz, 6-Me), 1.25 and 1.26 [2×s, in each case (3H), 2×Me], 5.79 (s, 4-H).

MS (EI+) m/z (rel. intensity)=323 (100); corresponds to C₂₂H₂₉NO.

### EXAMPLE 19

### 17β-Cyanoandrosta-4,6-dien-3-one

### 19a)

### 17β-Cyano-3-ethoxyandrost-3,5-diene

17β-Cyanoandrost-4-en-3-one was converted analogously to the method specified in Example 1a, except that trimethyl orthoformate was exchanged for triethyl orthoformate. 17β-Cyano-3-ethoxyandrost-3,5-diene was obtained.

¹H NMR (D6-DMSO): 0.81 (s, 3H), 0.86 (s, 3H), 1.17 (t, 3H, J=7.1 Hz, 3-O—CH2-CH3), 3.36 (m, 2H, 3-O—CH2-CH3), 5.09 (m, 2H, H-4 and H-6)

### 19b)

### 17β-Cyanoandrosta-4,6-dien-3-one

17β-Cyano-3-ethoxyandrost-3,5-diene was converted analogously to the method specified in Example 6. 17β-Cyanoandrosta-4,6-dien-3-one was obtained.

¹H NMR (300 MHz, CDCl₃, TMS as an internal standard, selected signals): δ=1.02 (s, 3H), 1.13 (s, 3H), 5.68 (s, 1H, H-4), 6.06 (s, 1H, 6-H), 6.13 (s, 1H, 7-H)

### EXAMPLE 20

### 17β-Cyano-7α-methylandrost-4-en-3-one

17β-Cyanoandrosta-4,6-dien-3-one was converted analogously to the method specified in Example 8. 17β-Cyano-7α-methylandrost-4-en-3-one was obtained.

¹H NMR (300 MHz, CDCl₃, TMS as an internal standard, selected signals): δ=0.77 (d, 3H, 7-CH3, J=7.3 Hz), 0.98 (s, 3H), 1.21 (s, 3H), 5.74 (s, 1H, H-4)

### EXAMPLE 21

17β-Cyano-7α-ethylandrost-4-en-3-one

17β-Cyanoandrosta-4,6-dien-3-one was converted analogously to the method specified in Example 8, except that ethylmagnesium bromide was employed instead of the methylmagnesium bromide used there. 17β-Cyano-7α-ethylandrost-4-en-3-one was obtained.

¹H NMR (300 MHz, CDCl₃, TMS as an internal standard, selected signals): δ=0.88 (t, 3H, 7-CH2-CH3, J=7.3 Hz), 0.98 (s, 3H), 1.22 (s, 3H), 5.74 (s, 1H, H-4)

### EXAMPLE 22

### 17β-Cyano-6α,7α-methyleneandrost-4-en-3-one and 17β-cyano-6β,7β-methyleneandrost-4-en-3-one

17β-Cyanoandrosta-4,6-dien-3-one was converted analogously to the method specified in Example 7. 17β-Cyano-6α,7α-methyleneandrost-4-en-3-one and 17β-cyano-6β,7β-methyleneandrost-4-en-3-one were obtained.

### 17β-Cyano-6α,7α-methyleneandrost-4-en-3-one

¹H NMR (300 MHz, CDCl₃, TMS as an internal standard, selected signals): δ=0.46 (m, 1H), 0.77 (m, 1H), 0.85 (m, 1H), 1.01 (s, 3H), 1.15 (s, 3H), 5.95 (s, 1H, H-4)

### 17β-Cyano-6β,7β-methyleneandrost-4-en-3-one

¹H NMR (300 MHz, CDCl₃, TMS as an internal standard, selected signals): δ=0.79 (m, 1H), 0.95 (s, 3H), 1.09 (s, 3H), 6.01 (s, 1H, H-4)

### EXAMPLE 23

### 17β-Cyano-6β-hydroxymethylandrost-4-en-3-one

17β-Cyanoandrost-4-en-3-one was converted analogously to the method specified in Example 2. 17β-Cyano-6β-hydroxymethylandrost-4-en-3-one was obtained.

¹H NMR (300 MHz, CDCl₃, TMS as an internal standard, selected signals): δ=0.98 (s, 3H), 1.21 (s, 3H), 3.68 (m, 2H, 6-CH2-OH), 5.82 (s, 1H, H-4)

### EXAMPLE 24

### 17β-Cyano-6,6-ethylideneandrost-4-en-3-one

17β-Cyano-6β-hydroxymethylandrost-4-en-3-one was converted analogously to the examples specified in Examples 3a and 3b, except that the intermediate tosylate was converted further in crude form. 17β-Cyano-6,6-ethylideneandrost-4-en-3-one was obtained.

¹H NMR (300 MHz, CDCl₃, TMS as an internal standard, selected signals): δ=0.42 (m, 2H), 0.77 (m, 1H), 0.99 (s, 3H), 1.26 (s, 3H), 5.62 (s, 1H, H-4)

### EXAMPLE 25

### 17β-Cyano-17α-methylandrost-4-en-3-one

### 25a)

### 17-Cyano-3,3-ethanediyibisoxyandrost-5-ene

5 g of the cyano compound were stirred in a mixture of 56 ml of dichloromethane, 14 ml of ethylene glycol, 37 ml of trimethyl orthoformate and 1.5 g of para-toluenesulphonic acid at room temperature for two hours. After adding sodium hydrogencarbonate solution and ethyl acetate, the phases were separated and the organic phase was washed with water and saturated sodium chloride solution, dried over sodium sulphate, filtered and concentrated. The 17β-cyano-3,3-ethanedlylbisoxyandrost-5-ene thus obtained was used further without further purification.

### 25b)

### 17β-Cyano-3,3-ethanediylbisoxy-17α-methylandrost-5-ene

17β-Cyano-3,3-ethanediyibisoxyandrost-5-ene was converted analogously to the method specified in Example 9a. 17β-Cyano-3,3-ethanediylbisoxy-17α-methylandrost-5-ene was obtained.

¹H NMR (300 MHz, CDCl₃, TMS as an internal standard, selected signals): δ=1.04 (s broad, 6H), 1.28 (s, 3H), 3.94 (m, 4H, ketal), 5.34 (s, 1H, H-6)

### 25c)

### 17β-Cyano-17α-methylandrost-4-en-3-one

17β-Cyano-3,3-ethanediylbisoxy-17α-methylandrost-5-ene was converted analogously to the method specified in Example 1c. 17β-Cyano-17α-methylandrost-4-en-3-one was obtained.

¹H NMR (300 MHz, CDCl₃, TMS as an internal standard, selected signals): δ=1.09 (s, 3H), 1.20 (s, 3H), 1.28 (s, 3H), 5.73 (s, 1H, H-4)

### EXAMPLE 26

### 17β-Cyano-6β-hydroxymethyl-17α-methylandrost-4-en-3-one

17β-Cyano-17α-methylandrost-4-en-3-one was converted analogously to the method specified in Example 2. 17β-Cyano-6β-hydroxymethyl-17α-methylandrost-4-en-3-one was obtained.

¹H NMR (D6-DMSO): 1.01 (s, 3H), 1.15 (s, 3H), 1.25 (s, 3H), 3.35 (m, 1H, 6-CH2-OH), 3.57 (m, 1H, 6-CH2-OH), 4.73 (t, 1H, J=5.8 Hz, 6-CH2-OH), 5.65 (s, 1H, H-4)

### EXAMPLE 27

### 17β-Cyano-6,6-ethanediyl-17α-methylandrost-4-en-3-one

17β-Cyano-6β-hydroxymethyl-17α-methylandrost-4-en-3-one was converted analogously to the methods specified in Examples 3a and 3b, except that the intermediate tosylate was converted further in crude form. 17β-Cyano-6,6-ethanediyl-17α-methylandrost-4-en-3-one was obtained.

¹H NMR (300 MHz, CDCl₃, TMS as an internal standard, selected signals): δ=0.42 (m, 2H), 0.78 (m, 1H), 1.10 (s, 3H), 1.27 (s, 3H), 1.29 (s, 3H), 5.63 (s, 1H, H-4)

### EXAMPLE 28

### 17α-Allyl-17β-cyanoandrost-4-en-3-one

### 28a)

### 17β-Cyano-17α-methylandrost-4-en-3-one

17-Cyano-3,3-ethanediyibisoxyandrost-5-ene was converted analogously to the method specified in Example 9a, except that allyl bromide was used instead of the methyl iodide used there. 17α-Allyl-17β-cyano-3,3-ethanediylbisoxyandrost-5-ene was obtained.

¹H NMR (300 MHz, CDCl₃, TMS as an internal standard, selected signals): δ=1.10 (s, 3H), 1.13 (s, 3H), 3.99 (m, 4H, ketal), 5.26 (m, 2H, —CH═CH2), 5.39 (s, 1H, H-6), 5.97 (m, 1H, —CH═CH2)

### 28b)

### 17α-Allyl-17β-cyanoandrost-4-en-3-one

17α-Allyl-17β-cyano-3,3-ethanediyibisoxyandrost-5-ene was converted analogously to the method specified in Example 1c. 17α-Allyl-17β-cyanoandrost-4-en-3-one was obtained.

¹H NMR (300 MHz, CDCl₃, TMS as an internal standard, selected signals): δ=0.97 (m, 1H), 1.17 (s, 3H), 1.25 (5, 3H), 5.25 (m, 2H, —CH═CH2), 5.79 (s, 1H, 4-H), 5.96 (m, 1H, —CH═CH2)

### EXAMPLE 29

### 17β-Cyano-1α-methylandrost-4-en-3-one

### 29a)

### 17β-Cyanoandrost-1,4-dien-3-one

2.5 g of 17β-cyanoandrost-4-en-3-one and 2.7 g of dichlorodicyanobenzoquinone were boiled in 50 ml of dioxane for 3 hours. After cooling, the mixture was diluted with dichloromethane and filtered. The filtrate was washed with sodium hydrogen-carbonate solution, water and saturated sodium chloride solution. After drying over sodium sulphate, filtering and concentrating the filtrate, the mixture was chromatographed on silica gel with a hexane/ethyl acetate mixture. 17β-Cyanoandrost-1,4-dien-3-one was obtained.

¹H NMR (300 MHz, CDCl₃, TMS as an internal standard, selected signals): δ=1.00 (s, 3H), 1.25 (s, 3H), 6.07 (s, 1H, H-4), 6.25 (s broad, 1H, H-2), 7.06 (s, 1H, H-1)

### 29b)

### 17β-Cyano-1α-methylandrost-4-en-3-one

0.6 g of 17β-cyanoandrost-1,4-dien-3-one in 6 ml of tetrahydrofuran was admixed successively with 6 mg of copper(I) bromide, 1.1 ml of trimethylaluminium and 0.31 ml of trimethylsilyl chloride. After stirring at room temperature for three hours, the mixture was partitioned between water and ethyl acetate. The organic phase was washed successively with water and saturated sodium chloride solution, dried over sodium sulphate, filtered and concentrated. After chromatography on silica gel with a mixture of hexane/ethyl acetate, 17β-cyano-1α-methylandrost-4-en-3-one was obtained.

¹H NMR (300 MHz, CDCl₃, TMS as an internal standard, selected signals): δ=0.94 (d, 3H, 1-CH3), 0.98 (s, 3H), 1.29 (s, 3H), 5.71 (s, 1H, H-4)

### EXAMPLE 30

### 6β,7β-15β,16β-Bismethylene-17β-cyano-17α-methylandrost-4-en-3-one

### 30a)

### 6β,7β-15β,16β-Bismethylene-3β-tert-butyldimethylsilyloxy-5β-hydroxyandrostane-17-one

According to the method of Example 16a, 6β,7β-15β,16β-bismethylene-3β,5β-dihydroxyandrostan-17-one (Angew. Chemie 1982, 94, 718-719) and tert-butyldimethylsilyl chloride as the silylating reagent, after crystallization, afforded 6β,7β-15β,16β-bismethylene-3β-tert-butyldimethylsilyloxy-5β-hydroxyandrostan-17-one.

¹H NMR (300 MHz, CDCl₃, TMS as an internal standard, selected signals): δ=0.08 and 0.11 [2×s, in each case (3H), Si-Me], 4.13 (s, 3-H), 4.40 (s, 5-OH).

MS (Cl+) m/z (rel. intensity)=445 (50), 462 (15); corresponds to C₂₇H₄₄NO₃Si.

### 30b)

### 6β,7β-15β,16β-Bismethylene-3β-tert-butyldimethylsilyloxy-17-cyano-5β-hydroxyandrostan-17-one

6β,7β-15β,16β-Bismethylene-3β-tert-butyldimethylsilyloxy-5β-hydroxyandrostan-17-one was converted analogously to the method described in Example 1b. 6β,7β-15β,16β-Bismethylene-3β-tert-butyldimethylsilyloxy-17-cyano-5β-hydroxyandrostan-17-one was obtained as a mixture of the 17-epimeric nitriles, which were processed further without epimer separation.

¹H NMR (D6-DMSO): 0.02 (s, 3H), 0.04 (s, 3H), 0.40 (m, 2H), 0.60 (m, 1H), 0.74 (s, 3H), 0.82 (s broad, 12H), 2.36 (m, 2H), 2.97 (m, 1H), 4.01 (m, 1H)

### 30c)

### 6β,7β-15β,16β-Bismethylene-3β-5β-bishydroxy-17β-cyano-17α-methylandrostane

### 6β,7β-15β,16β-Bismethylene-3β-tert-butyldimethylsilyloxy-17-cyano-5β-hydroxyandrostan-17-one was converted analogously to the methods specified in Examples 9a and 16c. 6β,7β-15β,16β-Bismethylene-3β-5β-bishydroxy-17β-cyano-17α-methylandrostane was obtained.

¹H NMR (D6-DMSO): 0.40 (m, 2H), 0.61 (m, 1H), 0.74 (s, 3H), 0.93 (s, 3H), 1.36 (s, 3H), 1.93 (m, 1H), 2.03 (m, 1H), 3.79 (m, 1H)

### 30e)

### 6β,7β-15β,16β-Bismethylene-17β-cyano-17α-methylandrost-4-en-3-one

310 mg of 6β,7β-15β,16β-bismethylene-3β-5β-bishydroxy-17β-cyano-17α-methylandrostane were dissolved in 10 ml of acetone and admixed with 0.42 ml of Jones reagent. After 15 minutes, 0.4 ml of isopropanol was added to the mixture. Subsequently, the mixture was partitioned between water and ethyl acetate, and the organic phase was washed with saturated sodium chloride solution, dried over sodium sulphate, filtered and concentrated. Chromatography on silica gel with a mixture of hexane and ethyl acetate afforded 6β,7β-15β,16β-bismethylene-17β-cyano-17α-methylandrost-4-en-3-one.

¹H NMR (D6-DMSO): 0.41 (m, 1H), 0.85 (m, 1H), 0.99 (s, 3H), 1.02 (s, 3H), 1.31 (s, 3H), 5.86 (s, 1H, 4-H)

### EXAMPLE 31

### 17α-Allyl-6β,7β-15β,16β-bismethylene-17β-cyanoandrost-4-en-3-one

### 31a)

### 17α-Allyl-6β,7β-15β,16β-bismethylene-3β-5β-bishydroxy-17β-cyanoandrostane

6β,7β-15β,16β-Bismethylene-3β-tert-butyldimethylsilyloxy-17-cyano-5β-hydroxyandrostan-17-one was converted analogously to the methods specified in Examples 9a (replacement of the methyl iodide used there by allyl bromide) and 16c. 17α-Allyl-6β,7β-15β,16β-bismethylene-3β-5β-bishydroxy-17β-cyanoandrostane was obtained.

¹H NMR (D6-DMSO): 0.40 (m, 2H), 0.61 (m, 1H), 0.74 (s, 3H), 0.96 (s, 3H), 2.02 (m, 2H), 3.79 (m, 1H), 4.78 (m, 1H), 5.19 (s, 1H), 5.24 (m, 1H), 5.94 (m, 1H)

### 31b)

### 17α-Allyl-6β,7β-15β,16β-bismethylene-17β-cyanoandrost-4-en-3-one

17α-Allyl-6β,7β-15β,16β-bismethylene-3β-5β-bishydroxy-17β-cyanoandrostane was converted analogously to the method specified in Example 30e. 17α-Allyl-6β,7β-15β,16β-bismethylene-17β-cyanoandrost-4-en-3-one was obtained.

¹H NMR (D6-DMSO): 0.43 (m, 1H), 0.86 (m, 1H), 1.02 (s, 3H), 1.03 (s, 3H), 5.20 (m, 1H, —CH═CH2), 5.24 (m, 1H, —CH═CH2), 5.87 (s, 1H, 4-H), 5.94 (m, 1H, —CH═CH2)

### EXAMPLE 32

### 17β-Cyano-6-methyl-15β,16β-methyleneandrosta-4,6-dien-3-one

25 mg of Pd—C (10%, water-moist) are added to a solution of 100 mg of 17β-cyano-6-exo-methylene-15β,16β-methyleneandrost-4-en-3-one in 10 ml of ethanol, and heated to boiling. Subsequently, the solution of 0.5 ml of cyclohexene in 2 ml of ethanol is slowly added dropwise over 1 hour, and the mixture is heated at reflux for a further 7 hours. After cooling the reaction mixture, filtering off the catalyst and concentrating, 17β-cyano-6-methyl-15β,16β-methyleneandrosta-4,6-dien-3-one (91 mg) is obtained.

¹H NMR (400 MHz, CDCl₃, TMS as internal standard, selected signals): δ=0.53 [m, (1H) cyclopropyl], 2.78 [d, J=4.5 Hz, 17-H(α)], 5.89 and 6.18 [2×s, (each 1H), 4-H and 7-H].

MS (Cl+) m/z (rel. intensity)=322 (100), 339 (50): corresponds to C₂₂H₂₇NO.

### EXAMPLE 33

### 4-Chloro-17β-cyano-15β,16β-methyleneandrost-4-en-3-one

700 mg of 17β-cyano-15β,16β-methyleneandrost-4-en-3-one are dissolved in 8 ml of pyridine and cooled to 0° C. After adding 0.32 ml of sulphuryl chloride, the mixture is stirred at this temperature for 1.5 hours. After admixing with saturated aqueous sodium hydrogencarbonate solution, water and ethyl acetate, the phases are separated, and the organic phase is washed with water and saturated aqueous sodium chloride solution. After drying the organic phase over sodium sulphate and filtering, it is concentrated and the product is recrystallized from ethyl acetate. This gives 4-chloro-17β-cyano-15β,16β-methyleneandrost-4-en-3-one (211 mg).

¹H NMR (300 MHz, CDCl3, TMS as internal standard, selected signals): δ=0.47 [m. (1H) cyclopropyl], 2.75 [d, J=4.5 Hz, 17-H(α)], 3.33 (ddd, J₁=15.3 Hz, J₂=4.5 Hz, J₃=2.6 Hz).

### EXAMPLE 34

### 17β-Cyano-3-hydroxyimino-15β,16β-methyleneandrost-4-ene

700 mg of 17β-cyano-15β,16β-methyleneandrost-4-en-3-one are dissolved in 5 ml of pyridine and admixed with 211 mg of hydroxylamine hydrochloride. After stirring at bath temperature 125° C. for one hour, the mixture is partitioned between water and ethyl acetate. The organic phase is washed with water and saturated aqueous sodium chloride solution, dried over sodium sulphate, filtered and concentrated. Column chromatography afforded 17β-cyano-3-hydroxyimino-15β,16β-methyleneandrost-4-ene as an E/Z mixture of the oximes (157 mg).

¹H NMR (300 MHz, CDCl3, TMS as internal standard, selected signals): δ=0.43 [m, (1H) cyclopropyl], 2.73 [d, J=4.5 Hz, 17-H(α)], 3.05 (m, 5-H₁), 5.79 (m, 4-H).

MS (Cl+) m/z (rel. intensity)=325 (100), 342 (76); corresponds to C₂₁H₂₈N₂O.

### EXAMPLE 35

### 17β-Cyano-6-exomethylene-17α-methyl-15β,16β-methyleneandrost-4-en-3-one

According to the method of Example 4, 130 mg of 17β-cyano-6β-hydroxymethyl-17α-methyl-15β,16β-methyleneandrost-4-en-3-one are used to obtain, after chromatography on silica gel, 17β-Cyano-6-exomethylene-17α-methyl-15β,16β-methyleneandrost-4-en-3-one (86 mg).

17β-Cyano-6-exomethylene-17α-methyl-15β,16β-methyleneandrost-4-en-3-one

¹H NMR (300 MHz, CDCl₃, TMS as internal standard, selected signals): δ=0.47 [m, (1H) cyclopropyl], 1.12, 1.16 and 1.39 [3×s, (each 3H), 3×Me], 5.02 and 5.12 [2×t, J=2 Hz, (each 1H), ═CH₂], 5.93 (br. s, 4-H).

### EXAMPLE 36

### 17β-Cyano-17α-methyl-15β,16β-methyleneandrosta-4,6-dien-3-one

According to the method of Example 6, 17.7 g of 17β-cyano-3-methoxy-17α-methyl-15β,16β-methyleneandrosta-3(4),5(6)-diene are used to obtain, after crystallization, 17β-Cyano-17α-methyl-15β,16β-methyleneandrosta-4,6-dien-3-one (5.84 g).

¹H NMR (300 MHz, CDCl₃, TMS as internal standard, selected signals): δ=0.52 [m, (1H) cyclopropyl], 1.14, 1.21 and 1.39 [3×s, (each 3H), 3×Me], 5.70 (s, 4-H), 6.18 (dd, J₁=9.8 Hz, J₂=2.8 Hz, 6-H), 6.33 (dd, J₁=9.6 Hz, J₂=1.8 Hz, 7-H).

MS (Cl+) m/z (relative intensity)=322 (100), 339 (32); corresponds to C₂₂H₂₇NO₂.

### EXAMPLE 37

### 6β,7β-15β,16β-Bismethylene-17β-cyano-17α-methylandrost-4-en-3-one and 6α,7α-15β,16β-Bismethylene-17β-cyano-17α-methylandrost-4-en-3-one

According to the method of Example 7, 3.0 g of 17β-cyano-17α-methyl-15β,16β-methyleneandrosta-4,6-dien-3-one are used to obtain, after HPLC separation of the crude product on silica gel, 6α,7α-15β,16β-bismethylene-17β-cyano-17α-methyl-androst-4-en-3-one (475 mg) as the nonpolar fraction, and 6β,7β-15β,16β-bismethylene-17β-cyano-17α-methylandrost-4-en-3-one (1.2 g) as the polar fraction.

### Fraction 1: 6α,7α-15β,16β-bismethylene-17β-cyano-17α-methylandrost-4-en-3-one

¹H NMR (300 MHz, CDCl₃ TMS as internal standard, selected signals): δ=0.46, 0.57, 0.80 and 0.93 [4×m, (each 1H), 4×cyclopropyl], 1.15, 1.20 and 1.39 [3×s, (each 3H), 3×Me], 5.96 (s, 4-H).

### Fraction 2: 6β,7β-15β,16β-bismethylene-17β-cyano-17α-methylandrost-4-en-3-one

¹H NMR (300 MHz, CDCl₃ TMS as internal standard, selected signals): δ=0.50 and 0.87 [2×m, (each 1H), 2×cyclopropyl], 1.10, 1.14 and 1.41 [3×s, (each 3H), 3×Me], 6.02 (s, 4-H).

MS (Cl+) m/z (rel. Intensity)=336 (100), 353 (28); corresponds to C₂₃H₂₉NO.

### EXAMPLE 38

### 17β-Cyano-17α-ethyl-3-methoxy-15β,16β-methyleneandrosta-3(4),5(6)-diene and 17β-cyano-17α-ethyl-15β,16β-methyleneandrost-4-en-3-one

According to the method of Example 9a), 18.0 g of 17β-cyano-3-methoxy-15β,16β-methyleneandrosta-3(4),5(6)-diene, using ethyl iodide instead of methyl iodide, are used to obtain, after crystallization, 17β-cyano-17α-ethyl-3-methoxy-15β,16β-methyleneandrosta-3(4),5(6)-diene (6.85 g) and, after flash chromatography of the mother liquor, 17β-cyano-17α-ethyl-15β,16β-methyleneandrost-4-en-3-one (338 mg).

### 17β-Cyano-17α-ethyl-3-methoxy-15β,16β-methyleneandrosta-3(4),5(6)-diene

¹H NMR (300 MHz, CDCl₃ TMS as internal standard, selected signals): δ=0.42 [m, (1H), cyclopropyl], 1.00, and 1.17 [2×s, (each 3H), 2×Me], 1.21 (t, J=7.1 Hz, CH₂—CH₃), 3.58 [s, (3H), OMe], 5.14 (m, 4-H), 5.26 (m, 6-H).

MS (Cl+) m/z (rel. intensity)=352 (90), 369 (100); corresponds to C₂₄H₃₃NO.

### 17β-Cyano-17α-ethyl-15β,16β-methyleneandrost-4-en-3-one

¹H NMR (300 MHz, CDCl₃ TMS as internal standard, selected signals): δ=0.45 [m, (1H), cyclopropyl], 1.19 and 1.21 [2×s, (each 3H), 2×Me; partly obscured by 1.23 (t, J≅6.4 Hz, CH₂—CH₃)], 5.75 (s, 4-H).

MS (Cl+) m/z (rel. intensity)=338 (100), 355 (59); corresponds to C₂₃H₃₁NO.

### EXAMPLE 39

### 17β-Cyano-17α-ethyl-15β,16β-methyleneandrosta-4,6-dien-3-one

According to the method of Example 6, 6.0 g of 17β-cyano-17α-ethyl-3-methoxy-15β,16β-methyleneandrosta-3(4),5(6)-diene are used to obtain, after crystallization and subsequent flash chromatography of the mother liquor, 17β-cyano-17α-ethyl-15β,16β-methyleneandrosta-4,6-dien-3-one (4.87 g).

¹H NMR (300 MHz, CDCl₃ TMS as internal standard, selected signals): δ=0.59 [m, (1H), cyclopropyl], 1.18 and 1.29 [2×s, (each 3H), 2×Me; partly obscured by 1.27 (t, J=7.4 Hz, CH₂—CH₃)], 5.75 (s, 4-H), 6.22 (dd, J₁=9.8 Hz, J₂=2.8 Hz, 6-H), 6.38 (dd, J₁=9.6 Hz, J₂=1.9 Hz, 7-H).

MS (Cl+) m/z (rel. intensity)=336 (100), 353 (43); corresponds to C₂₃H₂₉NO.

### EXAMPLE 40

### 6β,7β-15β,16β-Bismethylene-17β-cyano-17α-ethylandrost-4-en-3-one and 6α,7α-15β,16β-bismethylene-17β-cyano-17α-ethylandrost-4-en-3-one

According to the method of Example 7, 2.5 g of 17β-cyano-17α-ethyl-15β,16β-methyleneandrosta-4,6-dien-3-one are used to obtain, after HPLC separation of the crude product on silica gel, 6α,7α-15β,16β-bismethylene-17β-cyano-17α-ethylandrost-4-en-3-one (290 mg) as the nonpolar fraction, and 6β,7β-15β,16β-bismethylene-17β-cyano-17α-ethylandrost-4-en-3-one (670 mg) as the polar fraction.

### Fraction 1: 6α,7α-15β,16β-Bismethylene-17β-cyano-17α-ethylandrost-4-en-3-one

¹H NMR (300 MHz, CDCl₃ TMS as internal standard, selected signals): δ=0.48, 0.53, 0.80 and 0.93 [4×m, (each 1H), 4×cyclopropyl], 1.16 and 1.23 [2×s, (each 3H), 2×Me; partly obscured by 1.22 (t, J=6.3 Hz, CH₂—CH₃)], 5.96 (s, 4-H).

### Fraction 2: 6β,7β-15β,16β-Bismethylene-17β-cyano-17α-ethylandrost-4-en-3-one

¹H NMR (300 MHz, CDCl₃ TMS as internal standard, selected signals): δ=0.50 and 0.86 [2×m, (each 1H), 2×cyclopropyl], 1.09 and 1.15 [2×s, (each 3H), 2×Me], 1.22 (t, J=7.3 Hz, CH₂—CH₃), 6.00 (s, 4-H).

### EXAMPLE 41

### 17β-Cyano-17α-ethyl-7β-methyl-15β,16β-methyleneandrost-4-en-3-one and 17β-cyano-17α-ethyl-7α-methyl-15β,16β-methyleneandrost-4-en-3-one

According to the method of Example 8, 1.0 g of 17β-cyano-17α-ethyl-15β,16β-methyleneandrosta-4,6-dien-3-one is used to obtain, after HPLC separation of the crude product, 17β-cyano-17α-ethyl-7α-methyl-15β,16β-methyleneandrost-4-en-3-one (165 mg) as the nonpolar fraction and 17β-cyano-17α-ethyl-7β-methyl-15β,16β-methyleneandrost-4-en-3-one (292 mg) as the polar fraction.

### 17β-Cyano-17α-ethyl-7α-methyl-15β,16β-methyleneandrost-4-en-3-one

¹H NMR (300 MHz, CDCl₃ TMS as internal standard, selected signals): δ=0.44 [m, (1H), cyclopropyl], 0.86 (d, J=7.2 Hz, 7-Me), 1.08 [m, (1H), cyclopropyl], 1.19 and 1.21 [2×s, (each 3H), 2×Me; partly obscured by 1.22 (t, J=7.4 Hz, CH₂—CH₃)], 5.75 (s, 4-H).

### 17β-Cyano-17α-ethyl-7β-methyl-15β,16β-methyleneandrost-4-en-3-one

¹H NMR (300 MHz, CDCl₃ TMS as internal standard, selected signals): δ=0.53 and 1.04 [2×m, (each 1H), 2×cyclopropyl], 1.16-1.26 (m, 7-Me, 2×Me, and CH₂—CH₃)], 5.73 (br. s, 4-H).

### EXAMPLE 42

### 17β-Cyanoandrost-4-ene

Androst-4-en-17-one (see, for example, Helv. Chim. Acta (45) 1962, 2575) is converted analogously to the method specified in Example 1b. After chromatography of the resulting crude product on silica gel with a mixture of ethyl acetate and n-hexane, the product-containing fractions are concentrated and rechromatographed by HPLC. In addition to 17α-cyanoandrost-4-ene, 17β-cyanoandrost-4-ene is obtained.

### 17β-Cyanoandrost-4-ene

¹H NMR (CDCl₃ TMS as internal standard, selected signals): δ=0.94 [s, 3H, —CH₃], 1.04 [s, 3H, —CH₃], 1.13 [m, 1H], 1.21 [m, 1H], 2.11 [m, 1H], 2.20 [m, 1H], 2.26 [m, 1H], 5.31 [s broad, 1H, 4-H]

### EXAMPLE 43

### 4-Chloro-17β-cyanoandrost-4-en-3-one

17β-Cyanoandrost-4-en-3-one is reacted and worked up analogously to the method specified in Example 33. This gives 4-chloro-17β-cyanoandrost-4-en-3-one.

¹H NMR (CDCl3 TMS as internal standard, selected signals): δ=0.98 [s, 3H, CH₃], 1.24 [s, 3H, CH₃], 2.58 [m, 1H, 17-H], 3.26 [ddd, J₁=15.1 Hz, J₂=4.0 Hz, J₃=2.6 Hz]

### EXAMPLE 44

### 6β,7β-15β,16β-Bismethylene-17β-cyanoandrost-1,4-dien-3-one

6β,7β-15β,16β-Bismethylene-17β-cyanoandrost-4-en-3-one is converted analogously to Example 29a to obtain 6β,7β-15β,16β-bismethylene-17β-cyanoandrost-1,4-dien-3-one.

¹H NMR (300 MHz, CDCl₃): δ=0.55 (m, 1H), 1.10 (s, 3H), 1.14 (s, 3H), 2.81 (d, 1H, H-17) 6.18 (m, 1H, H-2), 6.33 (m, 1H, H-4), 6.85 (s, 1H, H-1)

### EXAMPLE 45

### 1α,2α-6β,7β-15β,16β-Trismethylene-17β-cyanoandrost-4-en-3-one

6β,7β-15β,16β-Bismethylene-17β-cyanoandrost-1,4-dien-3-one is converted analogously to Example 7 to obtain 1α,2α-6β,7β-15β,16β-trismethylene-17β-cyanoandrost-4-en-3-one

¹H NMR (300 MHz, CDCl₃): δ=0.52 (m, 1H), 0.74 (m, 1H), 0.82 (m, 1H), 1.07 (s, 3H), 1.14 (s, 3H), 2.81 (d, 1H, H-17), 5.86 (m, 1H, H-4)

1. A 17β-Cyano-19-androst-4-ene compound according to formula 1 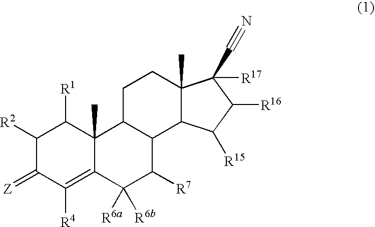
where
Z is selected from O, two hydrogen atoms, NOR and NNHSO₂R;
R is hydrogen or C₁-C₄-alkyl;
R¹, R² are each independently hydrogen or methyl, or R¹ and R² together form methylene or are omitted with formation of a double bond between C¹ and C²;
R⁴ is hydrogen or halogen;
and either:
R^{6a}, R^{6b} together form methylene or 1,2-ethanediyl, or R^{6a} is hydrogen and R^{6b} is selected from hydrogen, methyl and hydroxymethylene, and
R⁷ is selected from hydrogen, C₁-C₄-alkyl, C₂-C₃-alkenyl and cyclopropyl,
or:
R^{6a} is hydrogen, and
R^{6b} and R⁷ together form methylene or are omitted with formation of a double bond between C⁶ and C⁷
or:
R^{6a} is methyl and R^{6b} and R⁷ are omitted with formation of a double bond between C⁶ and C⁷;
R¹⁵, R¹⁶ are hydrogen or together form methylene;
R¹⁷ is selected from hydrogen, C₁-C₄-alkyl and allyl;
or a solvate, hydrate, stereoisomer, diastereomer, enantiomer or and salt thereof with the proviso that compounds of formula A are excluded: 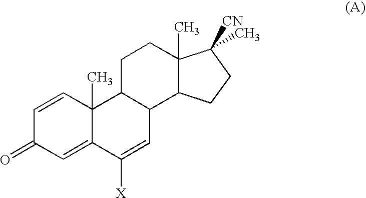
in which X is hydrogen or methyl and the double bonds between C¹ and C² and between C⁶ and C⁷ are optional double bonds, and
with the further proviso that 17β-cyanoandrost-4-en-3-one is also excluded.

2. A 17β-cyanoandrost-4-ene compound according to claim 1, wherein R¹⁵, R¹⁶ together form methylene.

3. A 17β-cyanoandrost-4-ene compound according to claim 1, wherein Z is selected from O, NOH and NNHSO₂H.

4. A 17β-cyanoandrost-4-ene compound according to claim 1, wherein Z represents O.

5. A 17β-cyanoandrost-4-ene compound according to claim 1, wherein R¹ and R² are in each case hydrogen or together form α-methylene.

6. A 17β-cyanoandrost-4-ene compound according to claim 1, wherein R¹ is α-methyl.

7. A 17β-cyanoandrost-4-ene compound according to claim 1, wherein R⁴ is hydrogen or chlorine.

8. A 17β-cyanoandrost-4-ene compound according to claim 1, wherein R^{6b} is methyl or hydroxymethyl.

9. A 17β-cyanoandrost-4-ene compound according to claim 1, wherein R^{6a}, R^{6b} together form methylene or 1,2-ethanediyl or are in each case hydrogen.

10. A 17β-cyanoandrost-4-ene compound according to claim 1, wherein R⁷ is selected from hydrogen, methyl and ethyl.

11. A 17β-cyanoandrost-4-ene compound according to one of claims 1 to 6, characterized in that R^{6b}, R⁷ together form methylene.

12. A 17β-cyanoandrost-4-ene compound according to claim 1, wherein R¹⁷ is selected from hydrogen, methyl and allyl.

13. A 17β-cyanoandrost-4-ene compound according to claim 1, wherein at least one of the substituents R¹, R², R⁴, R^{6a}, R^{6b}, R⁷, R¹⁵, R¹⁶ an d R¹⁷ is not hydrogen.

14. A 17β-cyanoandrost-4-ene compound according to claim 1, selected from:
6β,7β;15β,16βbismethylene-17β-cyano-17α-methylandrost-4-en-3-one,
6β,7β;15β,16β-bismethylene-17β-cyanoandrost-4-en-3-one,
17α-allyl-6β,7β;15β,16β-bismethylene-17β-cyanoandrost-4-en-3-one,
17β-cyanoandrost-4,6-dien-3-one,
17β-cyano-6β-hydroxymethyleneandrost-4-en-3-one,
17β-cyano-6α,7α-methyleneandrost-4-en-3-one,
17β-cyano-6β,7β-methyleneandrost-4-en-3-one,
17β-cyano-6,6-ethanediylandrost-4-en-3-one,
17α-allyl-17β-cyanoandrost-4-en-3-one,
17β-cyano-1α-methylandrost-4-en-3-one,
17β-cyanoandrost-1,4-dien-3-one,
17β-cyano-7α-ethylandrost-4-en-3-one,
17β-cyano-15β,16β-methyleneandrost-4-en-3-one,
17β-cyano-15α,16α-methyleneandrost-4-en-3-one,
17β-cyano-6β-hydroxymethylene-17α-methylandrost-4-en-3-one,
17β-cyano-6,6-ethanediyl-17α-methylandrost-4-en-3-one,
17β-cyano-6β-hydroxymethylene-15β,16β-methyleneandrost-4-en-3-one,
17β-cyano-17α-methyl-15β,16β-methyleneandrost-4-en-3-one,
17β-cyano-6,6-ethanediyl-15β,16β-methyleneandrost-4-en-3-one,
17α-allyl-17β-cyano-15β,16β-methyleneandrost-4-en-3-one,
17β-cyano-6,6-exo-methylene-15β,16β-methyleneandrost-4-en-3-one,
17β-cyano-6β-hydroxymethylene-15α-16α-methyleneandrost-4-en-3-one,
17β-cyano-6,6-exo-methylene-15α,16α-methyleneandrost-4-en-3-one,
17β-cyano-6,6-ethanediyl-15α,16α-methyleneandrost-4-en-3-one,
17β-cyano-15β,16β-methyleneandrost-4,6-dien-3-one,
17β-cyano-6α-methyl-15β,16β-methyleneandrost-4-en-3-one,
17β-cyano-17α-methyl-15α,16α-methyleneandrost-4-en-3-one,
17β-cyano-6β-hydroxymethylene-17α-methyl-15β,16β-methyleneandrost-4-en-3-one,
6α,7α;15β,16β-bismethylene-17β-cyanoandrost-4-en-3-one,
17β-cyano-15α,16α-methyleneandrost-4,6-dien-3-one,
17β-cyano-7α-methyl-15α,16α-methyleneandrost-4-en-3-one,
17β-cyano-6,6-exo-methylene-17α-methyl-15α,16α-methyleneandrost-4-en-3-one,
17β-cyano-7α-methylandrost-4-en-3-one,
17β-cyano-7α-methyl-15β,16β-methyleneandrost-4-en-3-one and
17β-cyano-6-methyl-15β,16β-methyleneandrost-4,6-dien-3-one.

15. A method of inducing oral contraception in a patient or treating pre-, peri- and postmenopausal symptoms in a patient, comprising administering to said patient a 17β-cyanoandrost-4-ene compound according to claim 1.

16. A method according to claim 15, wherein said compound has gestagenic and antimineralcorticoid action.

17. A pharmaceutical composition comprising at least one 17β-cyanoandrost-4-ene compound according to claim 1 and at least one suitable pharmaceutically harmless additive.

18. A pharmaceutical composition according to claim 17, further comprising at least one estrogen.

19. A pharmaceutical composition according to claim 18, wherein said estrogen is ethynylestradiol.

20. A pharmaceutical composition according to claim 18, wherein said estrogen is a natural estrogen.

21. A pharmaceutical composition according to claim 20, wherein the natural estrogen is estradiol.

22. A pharmaceutical composition according to claim 20, wherein the natural estrogen is estradiol valerate.

23. A pharmaceutical composition according to claim 20, wherein the natural estrogen is a conjugated estrogen.
